SEQUENCE LISTING (26 sequences)

SEQ ID NO: 1 - protein, 6 aa, Homo sapiens
CSVTCG

SEQ ID NO: 2 - protein, 380 aa, Homo sapiens
  Xaa represents an unknown amino acid
MVLESTMVCVDNSEYMRNGDFLPTRLQAQQDAVNIVCHSKTRSNPENNVGLITLANDCEVLTTLTPDTGRILSKLHTVQPKGKITFCTGIRVAHLALKHRQGKNHKMRIIAFVGSPVEDNEKDLVKLAKRLKKEKVNVDIINFGEEEVNTEKLTAFVNTLNGKDGTGSHLVTVPPGPSLADALISSPILAGEGGAMLGLGASDFEFGVDPSADPELALALRVSMEEQRQRQEEEARRAAAASAAEAGIATTGTEGERDSDDALLKMTISQQEFGRTGLPDLSSMTEEEQIAYAMQMSLQGAEFGQAESADIDASSAMDTSEPAKEEDDYDVXQDPEFLQSVLENLPGVDPNNEAIRNAMGSLASQATKDGKKDKKEEDKK

SEQ ID NO: 3 - protein, 377 aa, Homo sapiens
  Xaa represents an unknown amino acid
MVLESTMVCVDNSEYMRNGDFLPTRLQAQQDAVNIVCHSKTRSNPENNVGLITLANDCEVLTTLTPDTGRILSKLHTVQPKGKITFCTGIRVAHLALKHRQGKNHKMRIIAFVGSPVEDNEKDLVKLAKRLKKEKVNVDIINFGEEEVNTEKLTAFVNTLNGKDGTGSHLVTVPPGPSLADALISSPILAGEGGAMLGLGASDFEFGVDPSADPELALALRVSMEEQRQRQEEEARRAAAASAAEAGIATTGTEDSDDALLKMTISQQEFGRTGLPDLSSMTEEEQIAYAMQMSLQGAEFGQAESADIDASSAMDTSEPAKEEDDYDVXQDPEFLQSVLENLPGVDPNNEAIRNAMGSLASQATKDGKKDKKEEDKK

SEQ ID NO: 4 - DNA, 1259 bp, Homo sapiens
  CDS (1)..(1140): n/Xaa represents an unknown base/amino acid
atggtgttggaaagcactatggtgtgtgtggacaacagtgagtatatgcggaatggagacttcttacccaccaggctgcaggcccagcaggatgctgtcaacatagtttgtcattcaaagacccgcagcaaccctgagaacaacgtgggccttatcacactggctaatgactgtgaagtgctgaccacactcaccccagacactggccgtatcctgtccaagctacatactgtccaacccaagggcaagatcaccttctgcacgggcatccgcgtggcccatctggctctgaagcaccgacaaggcaagaatcacaagatgcgcatcattgcctttgtgggaagcccagtggaggacaatgagaaggatctggtgaaactggctaaacgcctcaagaaggagaaagtaaatgttgacattatcaattttggggaagaggaggtgaacacagaaaagctgacagcctttgtaaacacgttgaatggcaaagatggaaccggttctcatctggtgacagtgcctcctgggcccagtttggctgatgctctcatcagttctccgattttggctggtgaaggtggtgccatgctgggtcttggtgccagtgactttgaatttggagtagatcccagtgctgatcctgagctggccttggcccttcgtgtatctatggaagagcagcggcagcggcaggaggaggaggcccggcgggcagctgcagcttctgctgctgaggccgggattgctacgactgggactgaaggtgaaagagactcagacgatgccctgctgaagatgaccatcagccagcaagagtttggccgcactgggcttcctgacctaagcagtatgactgaggaagagcagattgcttatgccatgcagatgtccctgcagggagcagagtttggccaggcggaatcagcagacattgatgccagctcagctatggacacatccgagccagccaaggaggaggatgattacgacgtgatncaggaccccgagttccttcagagtgtcctagagaacctcccaggtgtggatcccaacaatgaagccattcgaaatgctatgggctccctggcctcccaggccaccaaggacggcaagaaggacaagaaggaggaagacaagaagtgagactggagggaaagggtagctgagtctgcttaggggactgcatgggaagcacggaatatagggttagatgtgtgttatctgtaaccattacagcctaaataaagcttggcaacttt

SEQ ID NO: 5 - DNA, 1250 bp, Homo sapiens
  CDS (1)..(1131): n/Xaa represents an unknown base/amino acid
atggtgttggaaagcactatggtgtgtgtggacaacagtgagtatatgcggaatggagacttcttacccaccaggctgcaggcccagcaggatgctgtcaacatagtttgtcattcaaagacccgcagcaaccctgagaacaacgtgggccttatcacactggctaatgactgtgaagtgctgaccacactcaccccagacactggccgtatcctgtccaagctacatactgtccaacccaagggcaagatcaccttctgcacgggcatccgcgtggcccatctggctctgaagcaccgacaaggcaagaatcacaagatgcgcatcattgcctttgtgggaagcccagtggaggacaatgagaaggatctggtgaaactggctaaacgcctcaagaaggagaaagtaaatgttgacattatcaattttggggaagaggaggtgaacacagaaaagctgacagcctttgtaaacacgttgaatggcaaagatggaaccggttctcatctggtgacagtgcctcctgggcccagtttggctgatgctctcatcagttctccgattttggctggtgaaggtggtgccatgctgggtcttggtgccagtgactttgaatttggagtagatcccagtgctgatcctgagctggccttggcccttcgtgtatctatggaagagcagcggcagcggcaggaggaggaggcccggcgggcagctgcagcttctgctgctgaggccgggattgctacgactgggactgaagactcagacgatgccctgctgaagatgaccatcagccagcaagagtttggccgcactgggcttcctgacctaagcagtatgactgaggaagagcagattgcttatgccatgcagatgtccctgcagggagcagagtttggccaggcggaatcagcagacattgatgccagctcagctatggacacatccgagccagccaaggaggaggatgattacgacgtgatncaggaccccgagttccttcagagtgtcctagagaacctcccaggtgtggatcccaacaatgaagccattcgaaatgctatgggctccctggcctcccaggccaccaaggacggcaagaaggacaagaaggaggaagacaagaagtgagactggagggaaagggtagctgagtctgcttaggggactgcatgggaagcacggaatatagggttagatgtgtgttatctgtaaccattacagcctaaataaagcttggcaacttt

SEQ ID NO: 6 - protein, 6 aa, Artificial Sequence
  Description of Artificial Sequence synthetic peptide
CSVTCG

SEQ ID NO: 7 - protein, 6 aa, Artificial Sequence
  Description of Artificial Sequence synthetic peptide
VCTGSC

SEQ ID NO: 8 - protein, 7 aa, Artificial Sequence
  Description of Artificial Sequence synthetic peptide
VCHSKTR

SEQ ID NO: 9 - protein, 7 aa, Artificial Sequence
  Description of Artificial Sequence synthetic peptide
VCHSKTR

SEQ ID NO: 10 - protein, 5 aa, Artificial Sequence
  Description of Artificial Sequence synthetic peptide
PHSRN

SEQ ID NO: 11 - protein, 6 aa, Artificial Sequence
  Description of Artificial Sequence synthetic peptide
ASVTAR

SEQ ID NO: 12 - DNA, 27 bp, Artificial Sequence
  Description of Artificial Sequence Primer
gggagatctatggtgttggaaagcact

SEQ ID NO: 13 - DNA, 27 bp, Artificial Sequence
  Description of Artificial Sequence Primer
ggggaattctcacttcttgtcttcctc

SEQ ID NO: 14 - protein, 7 aa, Artificial Sequence
  Description of Artificial Sequence synthetic peptide
KVWVLPI

SEQ ID NO: 15 - protein, 6 aa, Artificial Sequence
  Description of Artificial Sequence synthetic peptide
VCTGSC

SEQ ID NO: 16 - protein, 7 aa, Artificial Sequence
  Description of Artificial Sequence synthetic peptide
KSWVIPQ

SEQ ID NO: 17 - protein, 7 aa, Artificial Sequence
  Description of Artificial Sequence synthetic peptide
KLWVIPQ

SEQ ID NO: 18 - protein, 7 aa, Artificial Sequence
  Description of Artificial Sequence synthetic peptide
KVWVLPI

SEQ ID NO: 19 - protein, 7 aa, Artificial Sequence
  Description of Artificial Sequence synthetic peptide
KVWVLIP

SEQ ID NO: 20 - protein, 7 aa, Artificial Sequence
  Description of Artificial Sequence synthetic peptide
KVWIVST

SEQ ID NO: 21 - protein, 7 aa, Artificial Sequence
  Description of Artificial Sequence synthetic peptide
VCTGSCG

SEQ ID NO: 22 - protein, 6 aa, Artificial Sequence
  Description of Artificial Sequence synthetic peptide
CSVTCG

SEQ ID NO: 23 - protein, 6 aa, Artificial Sequence
  Description of Artificial Sequence synthetic peptide
GCTVSC

SEQ ID NO: 24 - protein, 132 aa, Homo sapiens
MVLESTMVCVDNSEYMRNGDFLPTRLQAQQDAVNIVCHSKTRSNPENNVGLITLANDCEVLTTLTPDTGRILSKLHTVQPKGKITFCTGIRVAHLALKHRQGKNHKMRIIAFVGSPVEDNEKDLVKLAKRLK

SEQ ID NO: 25 - protein, 133 aa, Homo sapiens
  Xaa represents an unknown amino acid
IATTGTEGERDSDDALLKMTISQQEFGRTGLPDLSSMTEEEQIAYAMQMSLQGAEFGQAESADIDASSAMDTSEPAKEEDDYDVXQDPEFLQSVLENLPGVDPNNEAIRNAMGSLASQATKDGKKDKKEEDKK

SEQ ID NO: 26 - protein, 7 aa, Artificial Sequence
  Description of Artificial Sequence synthetic peptide
VCTGSCG

(19) United States
(12) Patent Application Publication
(10) Pub. No.: US 2003/0180295 A1
(43) Pub. Date: Sep. 25, 2003

(54) Angiocidin: a Cys-Ser-Val-Thr-Cys-Gly specific tumor cell adhesion receptor

(75) Inventors: George Tuszynski, Pittsgrove, NJ (US); Taffy Williams, Lansdale, PA (US)

Correspondence Address: FINNEGAN, HENDERSON, FARABOW, GARRETT & DUNNER, LLP, 1300 I STREET, NW, WASHINGTON, DC, 20005, US

(73) Assignee: Philadelphia Health and Education Corporation

(21) Appl. No.: 10/122,348

(22) Filed: Apr. 16, 2002

Related U.S. Application Data
(62) Division of application No. 09/597,845, filed on Jun. 20, 2000, now abandoned.
(60) Provisional application No. 60/140,309, filed on Jun. 21, 1999.
(60) Provisional application No. 60/176,626, filed on Jan. 19, 2000.

(51) Int. Cl. A61K 39/395; C07K 14/705

(52) U.S. Cl. 424/143.1; 530/350

(57) ABSTRACT

The present invention provides the sequence of a cell matrix receptor specific for the Cys-Ser-Val-Thr-Cys-Gly (SEQ ID NO: 1) region of thrombospondin. Also provided are purification, cloning and expression methods. The receptor protein is useful in numerous diagnostic, prophylactic and therapeutic areas.

### PRIORITY INFORMATION

This application is a divisional of Ser. No. 09/597,845, filed Jun. 20, 2000, which is herein incorporated by reference, and claims the benefit of priority to two U.S. Provisional Application Serial No. 60/140,309, filed Jun. 21, 1999, and Serial No. 60/176,626, filed Jan. 19, 2000.

### TECHNICAL FIELD

Angiocidin, a cell matrix receptor, specific for the Cys-Ser-Val-Thr-Cys-Gly (SEQ ID NO: 1) region of thrombospondin expressed on the surface of tumor cells, is provided along with methods for purifying angiocidin and antibodies and inhibitors to angiocidin. Angiocidin is useful in numerous diagnostic and therapeutic conditions, such as cancer diagnosis, management, and treatment.

### BACKGROUND OF THE INVENTION

The mechanisms of cellular interaction with the basement membrane are of great interest because cancer cells must traverse the basement membrane before they can metastasize. The ubiquitous basement membrane is a specialized type of extracellular matrix separating organ parenchymal cells from interstitial collagenous stroma. Normal and neoplastic cells interact with this matrix differently. Most normal cells (nonmigratory ones) appear to require an extracellular matrix for survival, proliferation and differentiation, while migratory cells, both normal and neoplastic, must traverse the basement membrane in moving from one tissue to another. In particular, metastatic cancer cells arising in squamous or glandular epithelium traverse the basement membrane, entering the circulatory and lymphatic systems (intravasation). Circulating neoplastic cells are typically arrested in the capillary beds of another organ, invade the blood vessel walls, and penetrate the basement membrane to extravascular tissue (extravasation), where a secondary neoplasm is then established.

The interaction of cells with extracellular matrices is dependent upon the ability of the cells to attach themselves to the matrix. The attachment, in both normal and neoplastic cells, appears to be mediated by specific glycoproteins that bind cells to certain types of collagen proteins present in the matrix. For example, fibroblasts, myoblasts, and smooth muscle cells attach to the extracellular matrix through the interactions of fibronectin with interstitial type I and type III collagen, and chondrocytes attach through the interaction of chondronectin with type II cartilage collagen. Both normal and neoplastic cells attach to the basement membranes with similar mechanisms. The primary constituents of the basement membrane are type IV collagen, glycoproteins and proteoglycans. The glycoprotein laminin mediates the attachment of both epithelial and neoplastic cells to the basement membrane, binding the cells to type IV collagen.

Metastasizing tumor cells must traverse the basement membranes at multiple stages in the metastatic process, initiating this traversal by attaching to the basement membrane. Thus, elucidation of this mechanism and identification of specific attachment factors that promote or inhibit tumor cell attachment to this membrane has important implications for cancer diagnosis, prevention, management, and treatment.

Thrombospondin (TSP-1) is a cell adhesive protein and matrix molecule present in vascular basement membrane, tumor stroma, and is secreted by platelets. It mediates tumor cell invasion and metastasis. While not wishing to be bound by theory, it is believed that tumor cell colonization proceeds through the adhesive domain of TSP-1 containing the amino acid sequence Cys-Ser-Val-Thr-Cys-Gly (SEQ ID NO: 1), which binds to a novel Cys-Ser-Val-Thr-Cys-Gly (SEQ ID NO: 1)-specific tumor cell receptor, which has been named angiocidin. This receptor may be a transmembrane receptor, free, or cell associated.

TSP-1 is composed of three identical disulfide-linked chains each consisting of 1,152 amino acids (MW 145,000). Each polypeptide chain is composed primarily of domains consisting of repeating homologous amino acid sequences. These domains are an NH₂-terminal globular domain; a procollagen homology domain; the type 1 or properdin repeat domain, consisting of three repeating sequences homologous to sequences found in properdin; the type 2 repeat domain, consisting of three repeating sequences homologous to those in epidermal growth factor; the type 3 repeat domain, consisting of seven repeating Ca²⁺-binding sequences; and a COOH-terminal globular domain.

TSP-1 is characterized by the following activities, including cell-adhesion promoting activity, cell mitogenic activity, cell chemotactic activities, and hemostatic activities and any activities that derive from these activities such as tumor cell, microbial, or parasite metastasis activity, platelet aggregating activity, fibrinolytic activity and immune modulation.

Thrombospondin can bind to multiple cell surface receptors on the same cell or bind to different receptors on different cells, according to several studies. For example, platelets can bind TSP-1 through GPII b-IIIa, GPI a-IIa (Karczewski et al., J. Biol. Chem. 264:21332-21326 (1989) and Tuszynski et al., J. Clin. Invest. 87:1387-1394 (1991)), and the vitronectin-receptor (Tuszynski et al., Exp. Cell Res. 182:481 (1989)). Smooth muscle cells, endothelial cells, U937 monocyte-like cells, and melanoma cells can bind TSP-1 through a vitronectin-like receptor. Squamous cell carcinoma bind TSP-1 through a Mw 80,000/105,000 that is not an integrin or CD36. Yabkowitz et al., Cancer Res. 51:3648-3656 (1991).

The activity and importance of thrombospondin has been demonstrated by the function of antibodies developed against it. Antithrombospondin antibodies have been shown to inhibit platelet aggregation, confirming that thrombospondin plays a role in that system. Tuszynski et al., Blood 72:109-115 (1988). Additionally, antithrombospondin antibodies block cell adhesion to culture slides coated with thrombospondin, in contrast to slides with no antibody, which demonstrate cell adhesion. This provides further evidence that thrombospondin plays a role in cell adhesion and probably cancer metastasis. G. Tuszynski, Cancer Research 47:4130-33 (1987).

Receptors for other extracellular matrix proteins have been isolated. Liotta et al., U.S. Pat. No. 4,565,789, describe the isolation of a laminin receptor. Mecham et al., J. Biol. Chem. 264:16652-7 (1989), describe an elastin receptor which exhibits structural and functional similarity to the 67 kD laminin receptor. CD36 has been implicated as binding the Cys-Ser-Val-Thr-Cys-Gly (SEQ ID NO: 1) sequence of thrombospondin. Asch et al., Biochem. Biophys. Res. Comm. 182:1208-1217 (1992). However, CD36 is an 88 kD protein. The Cys-Ser-Val-Thr-Cys-Gly (SEQ ID NO: 1)-specific receptor of the present invention is different from these previously isolated extracellular matrix protein receptors.

All of the documents cited in this specification are incorporated herein by reference.

### SUMMARY OF THE INVENTION

It is an object of the present invention to provide purified receptors having specific binding affinity for the Cys-Ser-Val-Thr-Cys-Gly (SEQ ID NO: 1)-specific region of thrombospondin (TSP-1), preferably comprising a sequence selected from the group consisting of SEQ ID NO: 2 and SEQ ID NO: 3, fragments and mutations of SEQ ID NO: 2 and SEQ ID NO: 3, and antibodies and inhibitors to those receptors.

It is a further object of the invention to provide a method for treating or diagnosing disease using the receptor of SEQ ID NO: 2 and SEQ ID NO: 3, its fragments, mutants, or antibodies and ligands directed to it.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIGS. 1A and 1B (Sequence of Angiocidin) show the sequence of angiocidin, a Cys-Ser-Val-Thr-Cys-Gly-specific receptor protein (SEQ ID NO:2).

FIGS. 2A and 2B (Sequence of Angiocidin) show the sequence of angiocidin, a Cys-Ser-Val-Thr-Cys-Gly-specific receptor protein (SEQ ID NO:3).

FIGS. 3A and 3B (Sequence Comparison) compare the DNA sequence of the receptor identified in FIGS. 1A and 1B (SEQ ID NO:4) with the receptor identified in FIGS. 2A and 2B (SEQ ID NO:5).

FIG. 4 (Angiocidin SDS-PAGE gel) is an SDS-PAGE gel of angiocidin, the Cys-Ser-Val-Thr-Cys-Gly-specific receptor protein. Lane 1 is nonreduced protein (stained). Lane 2 is reduced protein (stained). Lane 3 is nonreduced protein (labeled). Lane 4 is reduced protein (labeled). Lane 5 is nonreduced surface-labeled protein.

FIG. 5 (Recombinant Angiocidin) is an analysis of recombinant receptor by SDS-PAGE and western blotting. Bacterial extracts containing expressed receptor, empty vector controls and purified his-receptor were analyzed by SDS-PAGE and blots stained with anti-receptor antibody. For Western blotting, membranes were treated with 1:2000 receptor antibody serum in TBS-tween (tris-buffered saline containing 0.05% Tween 20) for 2 hours, washed in TBS-tween, probed for 1 hour with 1: 15,000 horseradish peroxidase-conjugated anti-rabbit IgG, washed, and then revealed by ECL (Enhanced Chemilumi-nescence), Amersham, Arlington Heights, Ill. The various panels and lanes are as follows: Panel A, Stained gel, Panel B, anti-receptor antibody blot; and 1 Prestained MW standards, 2 Detergent bacterial extract with no insert, 3 Detergent bacterial extract with receptor insert, 4 Reduced his-tag purified receptor, 5 Nonreduced his-tag purified receptor, and 6 Prestained MW standards.

FIG. 6 (Binding of TSP-1 and Peptide to Angiocidin) shows the binding of TSP-1 and Cys(Acm)-Ser-Val-Thr-Cys(Acm)-Gly (SEQ ID NO: 6) to recombinant receptor. SDS-PAGE blots of bacterial lysates containing expressed receptor (lanes 2, 4, 7) or control lysates containing no expressed receptor (lanes 1, 3, 6) were either stained with anti-receptor antibody (lanes 1, 2), biotinylated TSP-1 (lanes 3, 4), or biotinylated Cys(Acm)-Ser-Val-Thr-Cys(Acm)-Gly (SEQ ID NO: 6) (lanes 6, 7).

FIG. 7 (Receptor Binding to Thrombospondin-1) shows the determination of receptor-TSP-1 binding constant. Binding of receptor to TSP-1 was determined by interaction analysis using the Affinity Sensor System, a resonant mirror biosensor system. TSP-1 was bound to a cuvette and receptor added. This figure shows a plot of the pseudo first order rate constant obtained from plots of instrument response vs time shown in FIG. 8.

FIG. 8 (Receptor Binding to Thrombospondin-1) shows the raw data used to determine the receptor-TSP-1 binding constant. Binding of receptor to TSP-1 was determined by interaction analysis using a resonant mirror biosensor system. This figure shows a sample instrument response vs time shown used to plot the data points in FIG. 7. The instrument response is proportional to the concentration of receptor-TSP-1 complex.

FIG. 9 (Effect of Receptor Peptides on Receptor Binding to TSP-1) shows the effect of receptor peptides on receptor binding to TSP-1 using the Affinity Sensor System, where the TSP-1 was bound to the cuvette and receptor binding measured. Receptor alone, and receptor plus a peptide (at two different molar ratios) were added. Receptor peptides, as well as a random negative control, were tested to measure their ability to inhibit the binding.

FIG. 10 (Binding of Receptor and Peptides to TSP-1) shows the binding of receptor alone as well as various peptides alone to immobilized TSP-1 on a cuvette. The receptor and the receptor peptides both bound to the TSP-1, while the random negative control peptide did not.

FIG. 11 (Receptor Binding to TSP-1 and Cys(Acm)-Ser-Val-Thr-Cys-(Acm)-Gly) shows that both TSP-1 and the peptide Cys(Acm)-Ser-Val-Thr-Cys(Acm)-Gly (SEQ ID NO: 6) bind to the receptor when the receptor is immobilized on a cuvette.

FIG. 12 (Localization of Receptor in Breast Tumors) shows the localization of receptor in breast tumors. The stained receptor can be visualized around the border of the tumor cells, found in the center of the figure.

FIG. 13 (Adhesion of Mock and Receptor Transfected Bovine Aortic Endothelial Cells) shows a cell adhesion study using receptor transfected cells binding to TSP-1 on a plate, or the negative control BSA. The receptor transfected cells adhered more strongly to the plate with TSP-1 than BSA.

FIG. 14 (Adhesion of B16-F10 Melanoma Cells to Receptor Peptides) shows a cell adhesion study with TSP-1, receptor peptides, and controls immobilized on a plate. The receptor transfected cells adhered strongly to the plates with fibronectin (positive control), TSP-1, and the receptor peptides. This may indicate that an additional protein is involved in the TSP-1 interaction.

FIG. 15 (Adhesion of TSP-1 Transfected MDA-MB 435 Breast Carcinoma Cells to Immobilized Recombinant Receptor) shows a cell adhesion study with TSP-1 transfected cells (and vector transfected control cells). The TSP-1 transfected cell bound more strongly to the receptor plate than the control cells.

FIG. 16 (Effect of Anti-TSP-1 Antibodies on Adhesion of TSP-1 Transfected MDA-MB-435 Breast Carcinoma Cells to Immobilized Recombinant Receptor) shows a cell adhesion study with TSP-1 transfected cells. This figure demonstrates that anti-TSP-1 and anti-Cys-Ser-Val-Thr-Cys-Gly (SEQ ID NO: 1) antibodies inhibited binding to the receptor covered plates.

FIG. 17 (Effect of Recombinant Receptor on Adhesion of MDA-MB-435 Breast Carcinoma) shows a cell adhesion study with TSP-1 transfected cells. The adhesion to receptor immobilized on a plate is inhibited by the addition of unbound TSP-1, in a concentration dependent fashion.

FIG. 18 (Effect of Receptor on Angiogenesis) shows the effect of angiocidin on angiogenesis. This figure demonstrates that angiocidin inhibited the formation of microtubules.

FIG. 19 (Effect of Receptor on Microvessel Stability) shows the effect of angiocidin on microvessel stability. This figure demonstrates that angiocidin broke up microtubules after formation in vitro.

FIG. 20 (Effect of Receptor on Morphology of Bovine Aortic Endothelial Cells) shows the effect of angiocidin on the morphology of bovine aortic endothelial cells. Increasing concentrations of angiocidin caused the cells to elongate, detach from the plate, aggregate, and die.

FIG. 21 (Effect of Receptor on Cell Viability) shows the effect of angiocidin on cell viability. BAEC and HUVEC cell lines have decreased viability in the presence of the receptor, suggesting that TSP is a requirement for viability of these cell lines. No significant difference was seen in the fibroblast, A549, MB231, and MCF7 cell lines, suggesting that TSP is not a requirement for viability in these cell lines.

FIG. 22 (Effect of Receptor on Viability of Bovine Aortic Endothelial Cells (BAEC) and Bovine Smooth Muscle Cells (BSM)) shows the effect of angiocidin on viability of BAEC and BSM cells. Angiocidin decreases viability of BAEC cells, but does not affect BSM cells.

FIG. 23 (Effect of Receptor on Viability of Bovine Aortic Endothelial Cells (BAEC) and Mouse Lewis Lung Carcinoma) shows the effect of angiocidin on viability of BAEC and mouse Lewis lung carcinoma cells. Angiocidin decreases viability of BAEC cells, but does not affect the Lewis lung cells.

FIG. 24 (Effect of Receptor on Viability of Human Umbilical Vein Endothelial Cells) shows the effect of angiocidin on viability of HUVEC cells, decreasing their viability.

FIG. 25 (Effect of Receptor on Viability of Human Umbilical Vein Endothelial Cells) shows the effect of angiocidin on viability of HUVEC cells, even in the presence of TSP-1.

FIG. 26 (Receptor-Mediated Viability of Bovine Aortic Endothelial Cells) shows the effect of angiocidin on viability of BAEC cells, even in the presence of TSP-1.

FIG. 27 (Receptor Binding Assay) presents a schematic representation of the biotin-avidin receptor binding assay.

FIG. 28 (Binding of Receptor to Immobilized TSP-1) illustrates the binding of angiocidin to immobilized TSP-1. This shows saturable binding, with a K_{D} of 9 nm.

FIG. 29 (Effect of Receptor on Binding of Biotin-Receptor to TSP-1) shows the competition effect of angiocidin on binding of the biotin-angiocidin complex to TSP-1.

FIG. 30 (Peptide Competition of TSP-1 Receptor Binding) shows the peptide competition of biotin-angiocidin complex binding to TSP-1 attached to the plate.

FIG. 31 (Receptor Binding Peptides From Phage Display Library) shows angiocidin-binding peptides from the phage display library screening process.

FIG. 32 (Peptide Competition (1 mg/ml) of TSP-1 Receptor Binding) shows peptide competition of TSP-1 and angiocidin binding. Both the Cys-Ser-Val-Thr-Cys-Gly (SEQ ID NO: 1) and Lys-Val-Trp-Val-Leu-Pro-IIe (SEQ ID NO: 14) peptides inhibit binding.

FIG. 33 (The Effect of Angiocidin on Viability of Human Aortic Endothelial Cells (HAEC) and Lung Human Microvascular Endothelial Cells (HMVEC-L)) shows the negative effect of angiocidin on viability of HAEC and HMVEC-L cells.

FIG. 34 (The Effect of Angiocidin and its Fragments on Viability of Bovine Aortic Endothelial Cells) shows the negative effect of angiocidin on BAEC cells, as well as the effect of various fragments of angiocidin.

FIG. 35 (The Effect of Angiocidin on Growth of Lewis Lung Carcinoma) qualitatively shows the in vivo effect of angiocidin on growth of Lewis lung carcinoma tumors in the flank of mice.

FIGS. 36A-36D illustrate the difference between control (FIGS. 36A and 36C) and angiocidin (FIGS. 36B and 36D) treated cells. The control cells are shown at 400× magnification under a light microscope. The angiocidin treated cells are shown at 200× magnification under a light microscope. The angiocidin treated cells show significant necrosis and cell death.

FIG. 37 (Effect of Angiocidin on Growth of Lewis Lung Carcinoma in vivo) quantitatively shows the in vivo effect of angiocidin on growth of Lewis lung carcinoma tumors in the flank of mice.

FIG. 38 (Effect of Angiocidin Treatment on Survival of Mice Bearing Lewis Lung Carcinoma) shows the effect of angiocidin treatment on survival of mice bearing Lewis lung carcinoma.

FIG. 39 (Viability Study) shows the effect of angiocidin on bovine aortic endothelial cell viability.

FIG. 40 (Effect of Anti-Angiocidin Antibody on Angiocidin-mediated Inhibition of BAEC Viability) shows the effect of anti-angiocidin antibody on angiocidin-mediated inhibition of bovine aortic endothelial cell viability.

FIG. 41 (Effect of Angiocidin on Adhesion of BAEC to a Substrate) shows the effect of angiocidin on the adhesion of bovine aortic endothelial cells.

FIG. 42 (Functionality of the Amino Terminal and Carboxy Terminal Portions of Angiocidin) shows that the N-terminal portion of the angiocidin protein contains all of the activity of the full length angiocidin protein, with respect to both TSP-1 binding and anti-endothelial activity. The C-terminal portion had activity levels similar to the negative control.

### DETAILED DESCRIPTION OF THE INVENTION

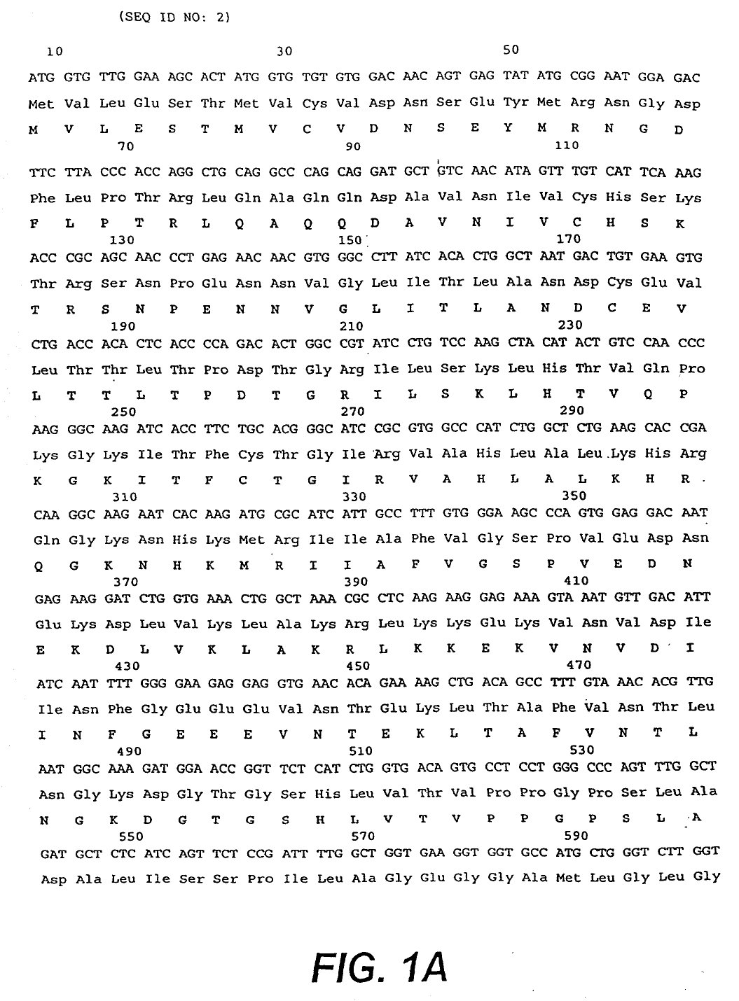

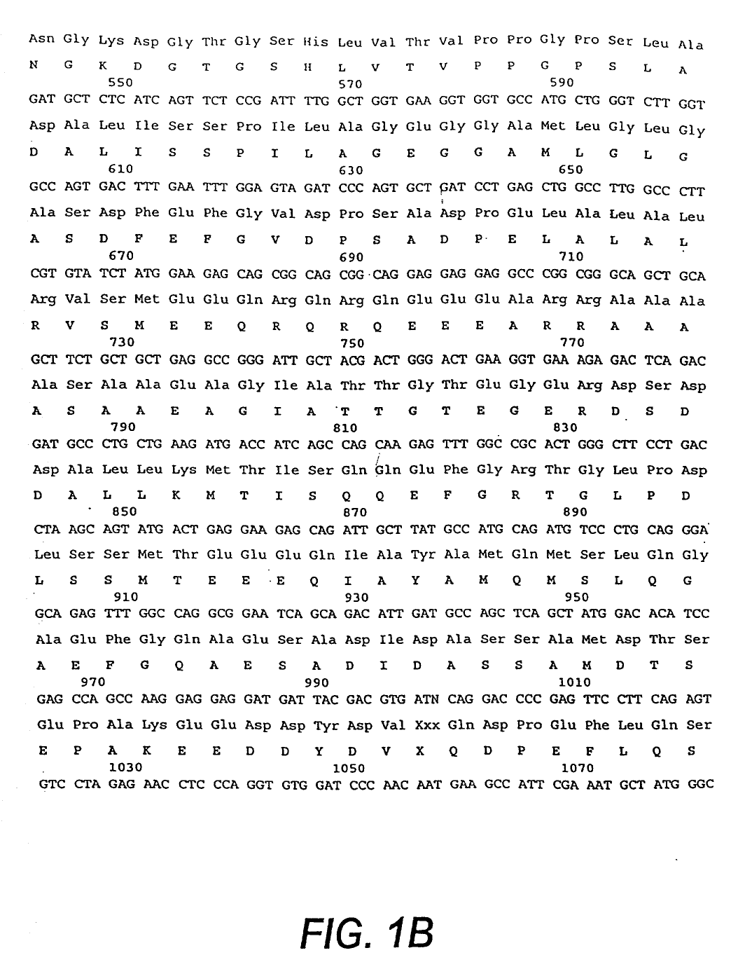

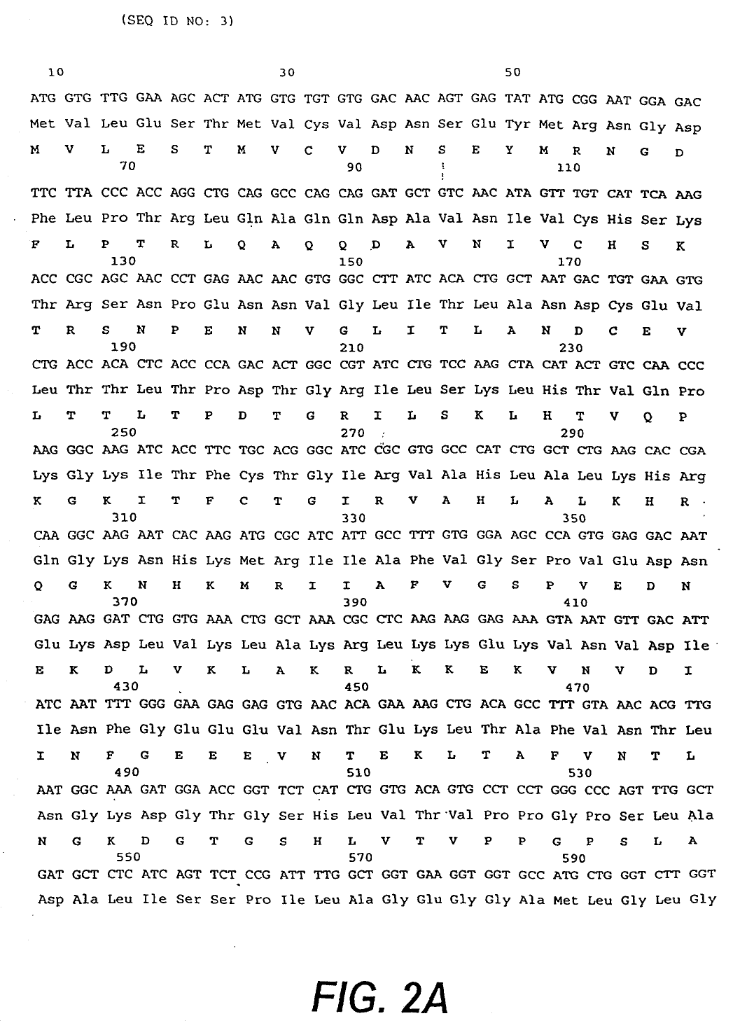

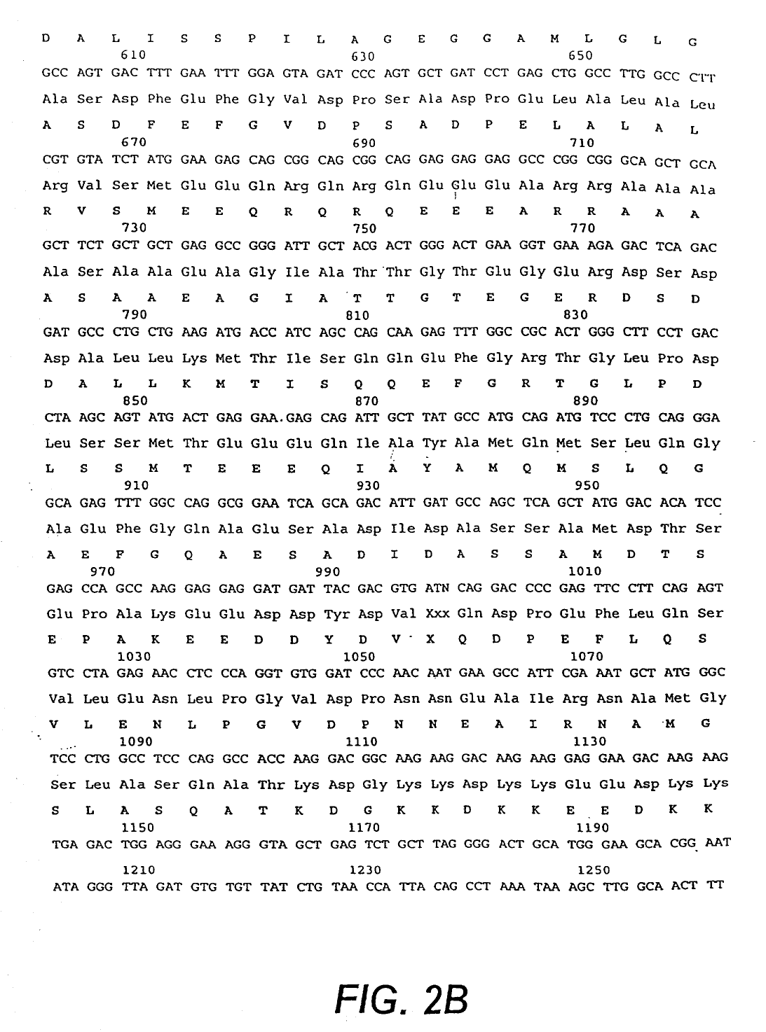

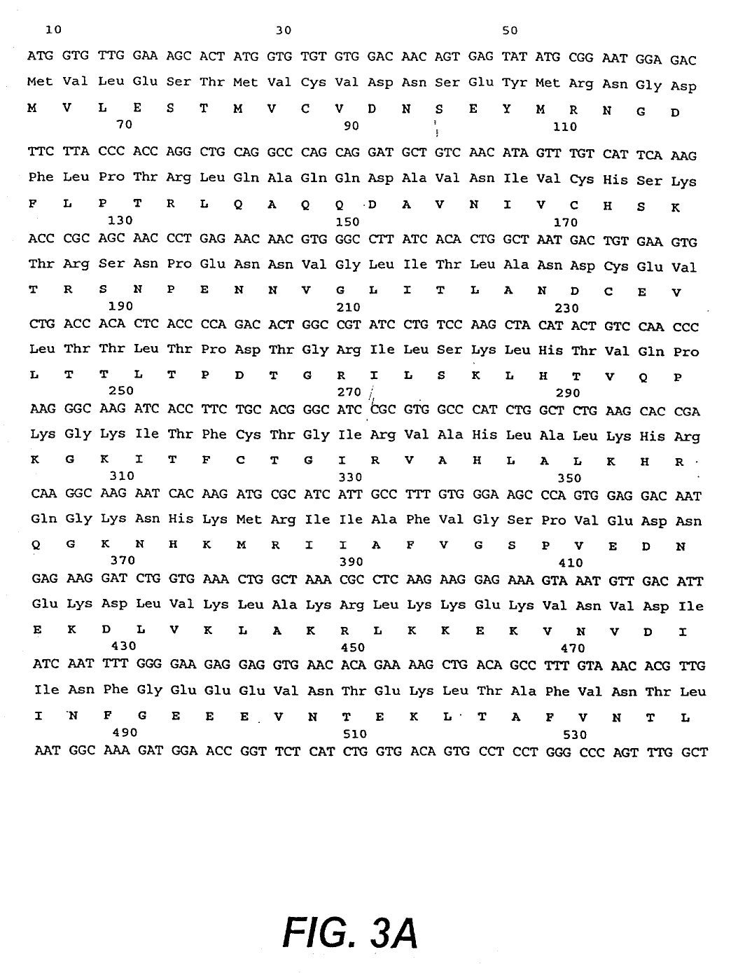

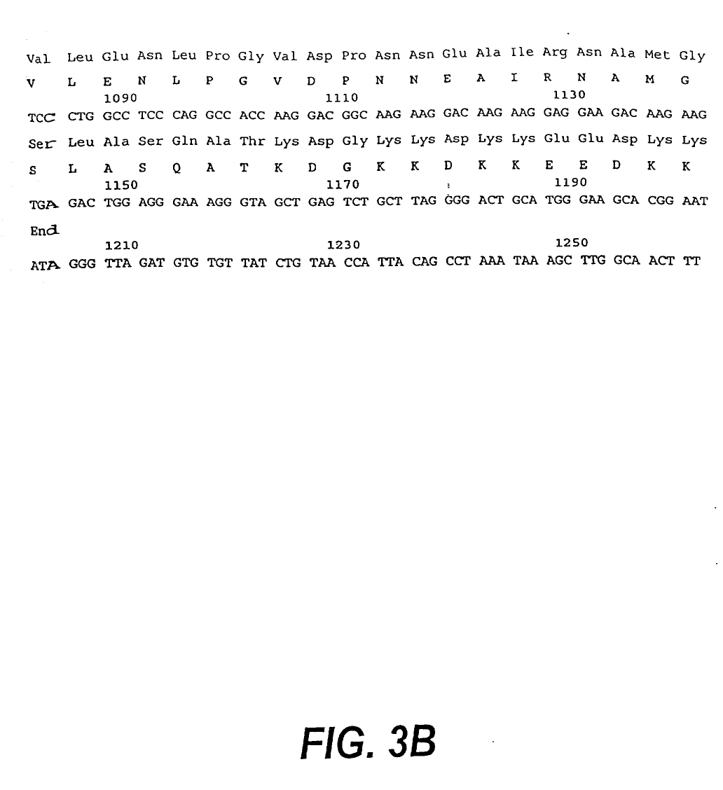

The present invention provides sequences of purified thrombospondin (TSP-1) receptor proteins, otherwise described herein as angiocidin. The sequences of the receptors can be found in FIGS. 1 and 2 (SEQ ID NO: 2 and SEQ ID NO: 3). The sequences differ by three amino acids Gly-Glu-Arg and the differences between their DNA sequences can be found in FIG. 3.

The receptors are specific for the Cys-Ser-Val-Thr-Cys-Gly (SEQ ID NO: 1) region of thrombospondin. The receptor proteins can be employed, for example, for producing antibodies which will be useful in numerous therapeutic areas, including cancer diagnosis or management. Computer modeling of the Cys-Ser-Val-Thr-Cys-Gly (SEQ ID NO: 1)-specific receptor binding site may also aid in the design of new compounds which block or bind the Cys-Ser-Val-Thr-Cys-Gly (SEQ ID NO: 1)-specific receptor site in vivo. This receptor protein is correlated with cancer and upregulated in cancer cells. This receptor is referred to herein as angiocidin.

The sequence of the receptor without the Gly-Glu-Arg (FIG. 2) shares sequence homology with two known, but unrelated proteins: antisecretory factor and the ubiquitin-binding subunit of human 26S protease. Antisecretory factor is a protein made by the pituitary and binds colonic epithelium and inhibits water transport into the colonic epithelium. Thus, this protein allows the body to regulate water flow in the gut. Antisecretory factor is produced under conditions of infection, such as when a host is infected by cholera. Johansson, E., Identification of an Active Site in the Antisecretory Factor Protein, Biochimica et Biophysica Acta 1362:177-82 (1997). The ubiquitin-binding subunit of human 26S protease, on the other hand, binds ubiquinated proteins and aids in the process of degrading old proteins in the cell. Ferrell, K., Molecular Cloning and Expression of a Multiubiquitin Chain Binding Subunit of the Human 26S Protease, FEBS Letters 381:143-48 (1996).

It is surprising that the thrombospondin receptor sequence shares sequence homology with both of these known proteins. Neither of these known proteins have been correlated with cancer or are known to be upregulated in cancer cells. The proteins do not share any function, and do not even act in the same regions of the body. The receptor of this invention is located on the cell surface, while antisecretory factor circulates in the blood, and the ubiquitin-binding subunit is contained within the cell. It is possible that the receptor may have different post-translational modifications from the two prior known proteins. These modifications may include: glycosylation, phosphorylation, ectophosphorylation, subunit structure (monomer vs. dimer or tetramer structure), and different conformational structures including binding of sulfhydryl groups.

It is believed that antibodies and ligands to the receptor of the present invention will not interfere with the actions of the antisecretory factor and the ubiquitin-binding subunit. The ubiquitin-binding subunit is located in an enzyme complex hidden within the cell and is likely to be protected from any cross-reactivity. Antisecretory factor appears to be produced in the body only under conditions of infection, specifically gastrointestinal infection. Thus, it is generally not present in the blood and thus, should not cross-react with antibodies to the receptor of this invention. Furthermore, the antibody specificity may be dependent on the post-translational modifications, which may be different between the three proteins. Addition of competitive receptor proteins similarly should not interfere with these other systems because of the likely post-translational differences between the proteins.

The receptors of the present invention also include receptors having modifications, otherwise known as mutations, of SEQ ID NO: 2 and SEQ ID NO: 3 that still allow binding to the thrombospondin peptide Cys-Ser-Val-Thr-Cys-Gly (SEQ ID NO: 1), with an affinity from about 106 M to about 10-10 M, preferably from about 10⁻⁷ M to about 10⁻⁹ M, most preferably about 10⁻⁸ M. The mutants may comprise any conservative substitutions that do not affect secondary structure or protein function, these include substitutions of amino acids in the same class such as hydrophobic, hydrophilic, basic, and acidic. Specifically, these include but are not limited to the following substitution pairs: valine and threonine, glycine and isoleucine, lysine and arginine, glutamic acid and aspartic acid, phenylalanine and tryptophan, serine and threonine, and methionine and cysteine. Preferentially, modifications are made to the carboxy terminal region, IIe248-Lys380 (SEQ ID NO: 25). This region seems not to affect the activity of angiocidin. However, modifications can be made to other regions as well. Other conservative substitutions would be readily apparent to the skilled artisan.

Additionally, fragments including the amino terminal region (Met1-Lys132) can be used in the present invention, as well as mutations of the fragments including the amino terminal fragment. The amino terminal fragment Met1-Lys132 can be found in SEQ ID NO: 24.

Definitions and Abbreviations

“Angiocidin,” “Cys-Ser-Val-Thr-Cys-Gly (SEQ ID NO: 1)-specific receptor protein,” “Thrombospondin receptor protein,” “TSP-1 receptor,” and “receptor” refer to a native thrombospondin receptor protein from any mammalian source, including, but not limited to, human, porcine, equine, bovine, and mouse which demonstrates a specific binding affinity for the peptide Cys-Ser-Val-Thr-Cys-Gly (SEQ ID NO: 1). This receptor has the sequence found in SEQ ID NO: 2 and SEQ ID NO: 3. The term also includes synthetic TSP-1 receptor protein, i.e., protein produced by recombinant means or direct chemical synthesis. TSP-1 receptor protein is a protein found in platelets, endothelial cells, epithelial (lung) cells, smooth muscle cells, fibroblasts, keratinocytes, monocyte macrophages, glial cells and most particularly cancer tissues, including, but not limited to, melanoma cells and lung carcinoma cells.

“Angiogenesis activity” is defined herein as the ability to inhibit or enhance the formation of blood vessels or lymph vessels.

“Anti-endothelial activity” is defined herein as the ability to decrease endothelial cell viability, such as bovine aortic endothelial cells.

“Antimalaria activity” is defined herein as the ability to inhibit either the cytoadherence of malarial-infected red blood cells to endothelial cells, the malarial sporozoite recognition and entry into hepatocytes, or the malarial merozoite recognition and entry into red blood cells. Antimalarial activity can be demonstrated in the form of a vaccine or a therapeutic that blocks cytoadherence.

and immune modulation.

“Antimetastatic activity” is defined herein as the ability to prevent or greatly reduce the extent or size of tumor cell metastasis, or inhibit or cause regression of primary solid tumors.

“Atherosclerosis activity” is defined herein as the capacity of thrombospondin to either promote or inhibit atherosclerotic lesion formation. The atherosclerotic lesion is defined as the degenerative accumulation of lipid-containing materials, especially in arterial walls.

“Cell adhesion activity” is defined herein as the ability to promote or inhibit the attachment of cells, preferably mammalian cells, to a substrate.

“Diabetic retinopathy activity” is defined herein as the ability to inhibit the abnormal formation of blood vessels in the eye caused by diabetes.

“Growth factor activity” is defined herein as the ability to inhibit or promote cell proliferation.

“Macular degeneration activity” is defined herein as the ability to inhibit the abnormal growth of blood vessels under the retina and macula in macular degeneration.

“Thrombospondin-like activity” is defined herein as any activity that mimics the known biological activities of thrombospondin. These activities include cell-adhesion promoting activity, cell mitogenic activity, cell chemotactic activities, and hemostatic activities and any activities that derive from these activities such as tumor cell, microbial, or parasite metastasis activity, platelet aggregating activity, fibrinolytic activity.

Preferred Embodiments

The preferred receptor proteins of the present invention have the sequences shown in FIGS. 1-2 (SEQ ID NO: 2 and SEQ ID NO: 3). Additional receptor proteins of the present invention also comprise mutants of those sequences, as described above. One preferred fragment covers the amino terminal (Met1-Lys1232) (SEQ ID NO: 24).

The Cys-Ser-Val-Thr-Cys-Gly (SEQ ID NO: 1)-specific receptor, angiocidin, is derived from cancer tissues, such as melanoma cells or lung carcinoma cells. Analysis of the receptor by sodium dodecylsulfate polyacrylamide gel electrophoresis (SDS-PAGE) shows that it has an apparent molecular weight of 50 kD under non-reducing conditions. In some preparations, small amounts of dimers could be observed with molecular weights of greater than 100 kD. Under reducing conditions, the protein migrates as two major polypeptide bands spaced closely together with apparent molecular weights of 50 and 60 kD, where the 50 kD species may be a degradation of the 60 kD species or a modified form. This is consistent with the interpretation that the protein consists of two interchain disulfide-linked polypeptide chains that assume a more compact configuration when disulfide bonded.

The protein does not cross react with antibodies against integrins, laminin, or CD 36. The Cys-Ser-Val-Thr-Cys-Gly (SEQ ID NO: 1)-specific receptor protein, angiocidin, is a glycoprotein since it binds galactose, mannose, and glucosamine specific lectins. Consistent with the presence of carbohydrate is the high 260 nm absorbance of the purified receptor protein.

To characterize the purified native angiocidin protein further its activity as a receptor in vitro was studied. The receptor interacts with thrombospondin in an ion dependent manner, but does not interact with fibronectin (FN) or bovine (BSA) serum albumin.

Use of Angiocidin

The TSP-1 receptors of this invention can be used in several ways. (1) Antibodies or ligands to the receptor can be generated. These antibodies or ligands can either mimic the effect of thrombospondin, or can interact with the receptor so as to block thrombospondin activity. (2) Knowledge of the receptor sequence can be used to measure a patient's receptor levels in blood, biopsy, or other tissue. Noninvasive tumors either do not express this receptor, or express it at only low levels, whereas invasive tumors express the receptor at high levels. The level of the receptor can indicate the patient's diagnosis or prognosis. This will provide a reliable tumor marker that will distinguish the noninvasive tumor cell, which may never spread, from the invasive phenotype, which metastasizes and causes mortality. This can help detect and treat malignant cancer. (3) The receptor can be used to design drugs to mimic or inhibit thrombospondin activity. (4) The receptor or fragments of the receptor may be administered to the patient as competitive inhibitors of thrombospondin activity. Modified forms of the receptor may be used instead of the receptor or its fragments. An acceptable fragment in this regard would preferably comprise the TSP-1 binding domain or a modification of this domain that binds to TSP-1 with an affinity from about 10⁻⁶ M to 10⁻¹⁰ M. (5) Cytotoxic drugs, hormones, imaging agents, or radioactive moieties can be coupled to an antibody or ligand directed to the receptor (which acts as a targeting moiety) for use in cancer treatment or other therapy. (6) A biomedical device can be coated with or linked to the antibodies to the receptor or ligand to the receptor to remove cells which bear receptors for thrombospondin on the cell surface, such as platelets. (7) The receptor or fragments of the receptor can be used to inhibit tumor growth, reduce the size of a tumor, or prevent tumor growth. (8) The receptor or fragments of the receptor can be used to prevent, inhibit, or reverse angiogenesis. One skilled in the art would understand other uses of the receptor of the present invention.

Any of these compositions can be administered to a patient along with nontoxic addition salts, amides and esters thereof, which may, alone, serve to provide the above-recited therapeutic benefits. Such compositions can also be provided together with physiologically tolerable liquid, gel or solid diluents, adjuvants and excipients. Standard formulations are known to those skilled in the art. Preferred modes of administration include intravenous, intramuscular, and subcutaneous administration. Another preferred mode of administration would direct the composition to the afflicted area(s) of the body, e.g., by linking the composition to a targeting agent. Additional formulations which are suitable for other modes of administration include suppositories, intranasal aerosols, and, in some cases, oral formulations.

For example, the antibodies of the present invention can mediate thrombospondin-like activity in a patient. One can use the antibodies of the present invention and compositions containing them, which have the physiological effect of inhibiting or mimicking the effect of intact thrombospondin, in numerous therapeutic and prophylactic applications, such as cancer therapy, atherosclerosis, malaria treatment or prevention, thrombotic or thrombolytic conditions, angiogenesis, or cell attachment. Antibodies are also useful as diagnostic reagents, therapeutics, or carriers of other compounds. The antibodies can also be used in biomedical devices.

These antibodies and compositions can be administered to animals for veterinary use, such as with domestic and farm animals or livestock, and clinical use in humans in a manner similar to other therapeutic antibody agents.

While not wishing to be bound by any theory, it is believed that the antibodies of the invention act as agonists or antagonists to native thrombospondin. These antibodies are also believed to act as agonists or antagonists to circumsporozoite protein, thrombospondin related anonymous protein, and properdin complement protein. Other ligands that contain the TSP-1 type 1 repeat sequences, such as METH-1 and METH-2 and related proteins belonging to the ADAMTS class of proteins, may interact with angiocidin. Vasquez, F., METH-1, a Human Ortholog of ADAMTS-1, and METH-2 are Members of a New Family of Proteins with Angio-Inhibitory Activity, J. Biol. Chem. 274:23349-23357 (1999). Ligands directed to the receptor can be used in the same way as the antibodies. The receptor or its fragments can also be administered as competitive ligands for thrombospondin. Mutants (i.e., modified forms of the receptor) of the receptor may also be administered as competitive ligands for thrombospondin.

Numerous in vitro and in vivo assays can be used to demonstrate that the antibodies effect thrombospondin-like activity. These assays include, but are not limited to: antibody-receptor binding assays, cell adhesion assays, platelet aggregation assays, and cell proliferation assays. A high throughput binding assay may be used, for example, to screen for antibodies to the receptor with thrombospondin-like binding. One can affix the receptor to a plate, bind labeled TSP-1, add the compound to be tested, and determine whether it inhibits TSP-1 binding to the receptor. Other assays, as discussed below, can be used to determine functional activity of the antibody to be tested.

Metastasis

Metastasis is the spread of disease from one part of the body to another unrelated to it, as in the transfer of the cells of a malignant tumor by way of the bloodstream or lymphatics. It is believed that metastasis is effected through a cascade mechanism which includes adhesion of tumor cells to endothelium, retraction of the endothelium, matrix degradation of the basement membrane and invasion of the tumor cells into the bloodstream. Intervention at any phase in this cascade could be beneficial to the treatment or prevention of metastatic cancers.

The native thrombospondin molecule has been shown to potentiate tumor cell metastasis. Tuszynski et al., Cancer Research, 47:4130-4133 (1987). The mechanisms by which the thrombospondin potentiation occurs are not presently well understood.

Antimetastatic activity is characterized by the ability of the compounds to bind to melanoma cells in vitro (Tuszynski et al., Anal. Bio., 184:189-91 (1990)), and the ability to reduce the size and number of tumor colonies in vivo (Tuszynski et al., Cancer Research, 47:4130-4133 (1987)).

Antibodies or ligands directed to the receptor are useful as antimetastatic agents, particularly useful as anti-pulmonary metastatic agents. These agents inhibit the adhesion of metastatic tumor cells, particularly those which are responsive to thrombospondin. They also reduce tumor colony number as well as tumor colony size. A particular advantage of the antibodies and the ligands are a long circulating half-life.

There are a number of mechanisms by which such antimetastatic activity can be occurring. The antibodies and ligands can be cytotoxic, or inhibit cell proliferation. As inhibitors of cell proliferation, these agents can act to 1) inhibit mitogenesis, 2) inhibit angiogenesis, or 3) activate the complement pathway and the associated killer cells. These mechanisms work by binding of the antibody or ligand to the receptor.

The antibodies and ligands of the invention can also find use in biomedical devices. Since the antibodies and ligands have the ability to promote the attachment of metastatic tumor cells, it is possible to coat a biomedical device with the agents to effect the removal of circulating tumor cells from blood or lymph. The biomedical device is also useful to trap hepatomas or other carcinomas.

Another use of the antibodies and ligands is as carriers to target toxins, drugs, hormones, imaging agents, or radioactive moieties to metastatic tumor cells for diagnostic or therapeutic purposes. These carriers would also bind to hepatomas or other carcinomas. The receptor itself, or its fragments/mutants can be used to competitively inhibit thrombospondin activity. Specifically, the invention includes a compositions and methods for treating cancer where the ligand or antibody directed to TSP-1 receptor is linked to a radioactive moiety. It also includes compositions and methods for radiological detection and diagnosis of cancer where the ligand or antibody directed to TSP-1 receptor is linked to a radioactive moiety. Radioactive moieties for treating, detecting, and diagnosing cancer are well known in the art. Lastly, it includes compositions and methods for MRI detection, diagnosis, and quantification of therapeutic response to treatement of cancer where the ligand or antibody directed to TSP-1 receptor is linked to an MRI enhancing agent. MRI enhancing agents for detecting, diagnosing, and quantifying therapeutic response of cancer are well known in the art, and include but are not limited to gadolinium, manganese, iron, technecium, GASTROGRAPHIN™, ISOVUE™, HEPATOLYTE™, and NEUROLYTE™. Other acceptable MRI enhancing agents would be known to the skilled artisan.

Atherosclerosis

Atherosclerosis is a disease state which is characterized by the deposition of small fatty nodules on the inner walls of the arteries, often accompanied by degeneration of the affected areas.

Administration of antibodies to the TSP-1 receptor, ligands to the TSP-1 receptor, or the receptor or its fragments/mutants can decrease thrombospondin activity and inhibit the development of aortic lesions. This result was demonstrated in rabbits fed a high cholesterol diet.

Diabetic Retinopathy

In diabetic retinopathy the blood vessels in the retina are damaged, leak fluid or bleed, causing retinal damage. In proliferative retinopathy, new, fragile blood vessels grow on the surface of the retina. These new blood vessels, or neovascularization, can lead to serious vision problems because they can break, leak, or bleed into the vitreous. As the vitreous becomes clouded with blood, light is prevented from passing through the eye into the retina, blurring or distorting vision. The new blood vessels can also cause scar tissue, which can pull the retina away from the back of the eye, causing retinal detachment. Retinal detachment leads to blindness. Lastly, abnormal blood vessels can grow on the iris, which can lead to glaucoma. It is believed that TSP may play a role in the abnormal blood vessel growth in diabetic retinopathy.

Macular Degeneration

In the “wet” type of macular degeneration, abnormal blood vessels (known as subretinal neovascularization) grow under the retina and macula. These new blood vessels may then bleed and leak fluid, thereby causing the macula to bulge or lift up, thus distorting or destroying central vision. Under these circumstances, vision loss may be rapid and severe. It is believed that TSP may play a role in the abnormal blood vessel growth in macular degeneration.

Malaria

Malaria is an infectious disease caused by any of various protozoans (genus Plasmodium) that are parasitic in the red blood corpuscles and are transmitted to mammals by the bite of an infected mosquito. The antibodies, ligands, or receptor or its fragments/mutants of the invention can be used as therapeutic agents to block cytoadherence.

These agents block thrombospondin activity and thus inhibit either the cytoadherence of malarial-infected red blood cells to endothelial cells, the malarial sporozoite recognition and entry into hepatocytes, or the malarial merozoite recognition and entry into red blood cells.

Angiogenesis

Angiogenesis is the formation of blood and lymph vessels. The antibodies, ligands, and receptors or its fragments/mutants of this invention are useful in the modulation of angiogenesis, particularly in enhancing wound healing, inhibiting or preventing tumor growth, diabetic retinopathy, macular degeneration and rheumatoid arthritis. Standard angiogenesis assays are well known in the art. These assays include, but are not limited to, proliferation and migration studies using various cell lines, collagenase inhibition and in vivo neovascularization on chicken chorioallantoic membranes (CAM assay).

Adhesion Modulation

The antibodies, ligands, and receptors or its fragments/mutants can modulate cell adhesion and inhibit binding of TSP-1 and other proteins to cells, such as blood platelets, which contain the TSP-1 receptor site.

Diagnostic

Antibodies and ligands of the invention can be useful as reagents in diagnostic/prognostic assays for various types of cancer, including but not limited to: gastrointestinal tract (gastric, colonic, and rectal) carcinomas, breast carcinomas, hepatic carcinomas, and melanomas. The level of the TSP-1 receptor can be used to provide patient prognosis or diagnosis. Further knowledge of the sequence of the receptor can be used directly to determine the level of the receptor in a patient sample.

Carrier

Cytotoxic drugs, hormones, imaging agents, or radioactive moieties can be coupled to the antibodies or ligands for use in cancer or other therapy.

Biomedical Device

A biomedical device can be coated with or linked to the antibodies or ligands to remove cells which bear receptors for thrombospondin on the cell surface, such as platelets.

Identification of Appropriate Ligands to the Thrombospondin Receptor

Appropriate ligands include the thrombospondin protein, its mutants and fragments (including the peptide Cys-Ser-Val-Thr-Cys-Gly (SEQ ID NO:1)), and other peptides or proteins that bind to the receptor of the present invention.

Such ligands can be developed and identified by using a phage display peptide library kit, such as that available from New England Biolabs (Beverly, Mass.). Phage display describes a selection technique in which a peptide or protein is expressed as a fusion with a coat protein of a bacteriophage, resulting in display of the fused protein on the exterior surface of the phage virion, while the DNA encoding the fusion resides within the virion. Phage display can be used to create a physical linkage between a vast library of random peptide sequences to the DNA encoding each sequence, allowing rapid identification of peptide ligands for a variety of target molecules (including receptors) by an in vitro selection process called biopanning. This technique is carried out by incubating a library of phage-displayed peptides with a plate (or bead) coated with the target receptor, washing away the unbound phage, and eluting the specifically-bound phage. The eluted phage is then amplified and taken through additional cycles of biopanning and amplification to successively enrich the pool of phage in favor of the tightest binding sequences. After 3-4 rounds, individual clones are characterized by DNA sequencing and ELISA.

The oligonucleotide encoding the peptide could then be used as a probe to identify proteins containing the identified peptide sequence. These proteins can then be evaluated for their binding capacity for the receptor using any of the binding techniques disclosed in the Examples below.

Expression of Angiocidin

Angiocidin, or any of its fragments or mutants, can be expressed in known expression systems, including mammalian cell lines, insect cells, yeast strains, and bacteria such as E. Coli.

Mammalian cell lines offer several advantages for expression of heterologous proteins. Eukaryotic proteins produced in mammalian cells will be functional since transcription, translation, and posttranslational modification processes are conserved among higher eukaryotes. Mammalian cell lines are well suited for a variety of recombinant protein studies including structure-function assays and analyzing the physiological effects of the protein on cell function.

Insect cells are an excellent host for recombinant protein expression. They are often chosen for protein production because as higher eukaryotes, they perform posttranslational modifications similar to mammalian cells, but grow faster and do not require CO₂ incubators. In addition, insect cells can be readily adapted to suspension culture for large scale expression.

Various yeast strains have proven to be extremely useful for expression and analysis of eukaryotic proteins. Yeast have been well characterized genetically and are known to perform many mammalian-like posttranslational modifications. These single-celled eukaryotic organisms grow quickly in defined medium, are easier and less expensive to work with than mammalian cells, and are easily adapted to fermentation. Yeast expression systems are therefore ideally suited for large-scale production of recombinant eukaryotic proteins.

Expression of recombinant proteins in E. coli. is rapid and offers high yields. However, the bacterial system may not produce optimally active protein since bacteria do not glycosylate proteins or optimally fold proteins. Nevertheless, bacterial expression systems are often preferred for their ease of use.

### EXAMPLES

The following examples are presented for illustrative purposes only and are not intended to limit the scope of the invention in any way. In the Examples using recombinant angiocidin, the sequence provided for in SEQ ID NO: 2 was used. Nevertheless, it is believed that the sequence provided for in SEQ ID NO: 3, as well as mutants and fragments of both sequences, would work effectively well in the invention.

### Example 1

### Purification of the Receptor

Purification of Cys-Ser-Val-Thr-Cys-Gly (SEQ ID NO: 1)-specific receptor protein from cells comprises two basic steps: preparation of the cells and purification of the receptor by affinity chromatography. Preferred cell sources included mouse melanoma cells and human lung carcinoma cells which are readily available to the public. Cultured cells have the additional benefit of being relatively protease-free compared to most tissue sources. This facilitates stabilization and purification of active receptor protein.

A cell extract can be prepared and passed through a chromatographic column containing immobilized Cys-Ser-Val-Thr-Cys-Gly (SEQ ID. NO: 1) peptides under conditions where the receptor will bind to the Cys-Ser-Val-Thr-Cys-Gly (SEQ ID NO: 1) peptide. The Cys-Ser-Val-Thr-Cys-Gly (SEQ ID NO: 1)-specific receptor is then eluted from the column in purified form.

Specifically, a cell extract was prepared from approximately 4.0×10⁷ B16-F10 mouse melanoma cells or A549 human lung carcinoma cells by dissolving the cell pellet in 5 ml of binding buffer (10 mM Tris-HCl, pH 7.5, containing 0.5% (NP)*-40 detergent, 1 mM CaCl₂, 1 mM MgCl₂, 100 μM leupeptin, 1 mM phenylmethyl sulfonyl fluoride (PMSF), 10 μg/ml aprotinin). Undissolved material was removed from the sample by centrifugation at 4,000×g for 20 min. at 4° C.

A Cys-Ser-Val-Thr-Cys-Gly (SEQ ID NO: 1) affinity column was constructed by packing a 5 ml column containing 4 mg of Cys-Ser-Val-Thr-Cys-Gly (SEQ ID NO: 1) coupled to 1 ml of CN-activated Sepharose equilibrated in HEPES buffered saline, pH 7.35. The extract was applied to the Cys-Ser-Val-Thr-Cys-Gly (SEQ ID NO: 1) column which had been washed with 50 ml of binding buffer. Nonspecifically adsorbed proteins were removed from the column by washing the column with 50 ml of binding buffer. Specifically adsorbed proteins were eluted with 0.10 M Tris, pH 10.2, containing 0.05% (NP)*-40 detergent, 1 mM CaCl₂, 1 mM MgCl₂, 100 μM leupeptin, 1 mM phenylmethyl sulfonyl fluoride (PMSF), and 10 μg/ml aprotinin. Ten ml fractions were collected in tubes containing 700 μl of 1 N HCl to neutralize the Tris. The peak fraction in tube was applied to an anion exchange column (Mono Q, Pharmacia) equilibrated in anion exchange column buffer (20 mM Tris HCl, pH 8.0, containing 5 mM octylglucoside). The bound material was eluted with a 20 ml gradient of NaCl (100% 1M NaCl) and the column monitored at 280 and 260 nm. The bound material routinely began to elute at 0.3M NaCl and the gradient was held to allow the proteins to elute isocratically yielding a single homogenous peak having a high absorbance at 260 nm.

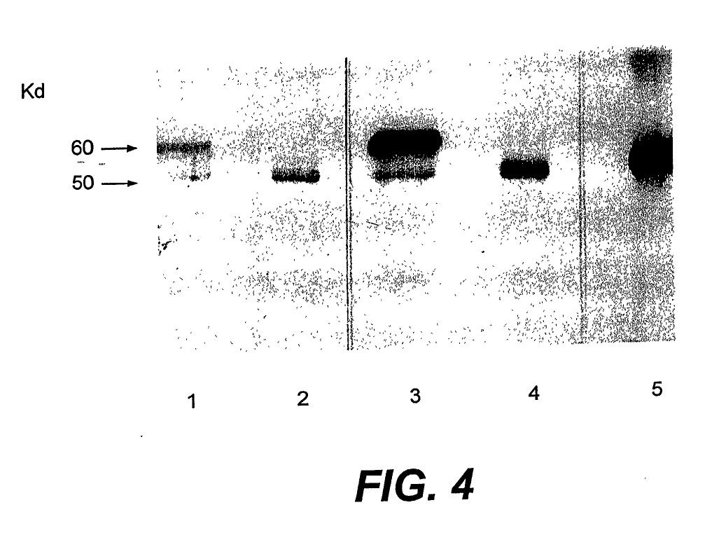

The eluted fraction and unbound fractions were concentrated and the concentrated material analyzed on SDS-gels on an 8% polyacrylamide gel and visualized by comassie blue stain using standard techniques. The peak fraction analyzed on SDS-gel electrophoresis under nonreducing conditions as a major band with an apparent molecular weight of 50 kD and under reducing conditions (5% beta-mercaptoethanol) as two polypeptide bands of 50 and 60 kD, as indicated in FIG. 4 (lanes 1 and 2). Approximately 100 μg of protein was recovered from 1×10⁷ cells. The Cys-Ser-Val-Thr-Cys-Gly (SEQ ID NO: 1)-specific receptor was labeled with ¹²⁵I-Iodine by the standard procedure of Karczewski et al., J. Biol. Chem. 264:21322-6 (1989). Briefly, 12 μg of purified protein dissolved in 100 μl of octylglucoside buffer was incubated with one Iodobead for 5 min. Unreacted iodide was removed on a small column of Sephadex G-25 equilibrated in octylglucoside buffer as previously described by Tuszynski et al., Anal. Biochem. 106:118-122 (1980). The specific activity of protein obtained in a typical experiment was 10⁴ cpm/pg. Analysis of the labeled material by SDS-gel electrophoreses followed by autoradiography indicated that under reducing conditions the 60 kD molecular weight polypeptide band was predominant. The autoradiogram of this labeled material is shown in FIG. 4, lanes 3 and 4.

### Example 2

### Molecular Cloning and Sequence Analysis of Cys-Ser-Val-Thr-Cys-Gly-Specific TSP-1 Receptor cDNA

The basic strategies for preparing antibodies or oligonucleotide probes and DNA libraries, as well as their screening by antibody or nucleic acid hybridization, are well known to those of ordinary skill in the art. See, e.g., DNA CLONING: VOLUME I (D. M. Glover ed. 1985): NUCLEIC ACID HYBRIDIZATION (B. D. Hames and S. J. Higgins eds. 1985): OLIGONUCLEOTIDE SYNTHESIS (M. J. Gate ed. 1984): T. Maniatis, E. F. Frisch & J. Sambrook, MOLECULAR CLONING: A LABORATORY MANUAL (11982). These known methods were followed for cloning and sequencing the receptor of the present invention.

Polyclonal antisera against receptor isolated from A549 human lung carcinoma was used to screen a lambda Uni-ZAP (Stratagene, La Jolla, Calif.) prostate cancer cell (PC3-NI) library kindly provided by Drs. Mark Stearns and Min Wang, MCP-Hahnemann University. Approximately, 200,000 plaques were screened with a 1:1000 dilution of anti-receptor antiserum adsorbed with phage and bacteria according to the procedure provided with the PicoBlue Immunostaining kit (Stratagene, LaJolla, Calif.). Four antibody positive plaques were isolated and cloned and phagemids were transferred to XL1 blue bacteria using the ExAssist Interference-Resistant Helper Phage protocol (Stratagene, LaJolla, Calif.). Plasmid DNA was purified using the Wizard plus miniprep (Promega, Madison, Wis.) and sequenced using the T7/T3 primer set by the dideoxy chain termination method with Sequenase version 2.0 (U.S. Biochemical Corp.). The resulting sequences can be found in FIGS. 1 and 2 (SEQ ID NO: 2 and SEQ ID NO: 3). The comparison of the DNA sequences for the two receptors can be found in FIG. 3 (SEQ ID NO: 4 and SEQ ID NO: 5).

### Example 3

### Expression of Recombinant Angiocidin

Full-length receptor cDNA subcloned in XL1-blue bacteria containing the PBK-CMV promoter were induced to express protein with IPTG (isopropyl-_D-thiogalactopyranoside) as described in current protocols in molecular biology. Bacteria were lysed with the B-Per bacterial Protein Extraction Reagent (Pierce Chemical Co Rockfort, Ill.).

The recombinant receptor can also be expressed in other bacterial, baculovirus, and mammalian cell (such as COS cells) expression systems. One skilled in the art would know that a bacterial system may not produce optimally active protein since bacteria do not glycosylate protein or optimally fold protein. The baculovirus expression system, however, produces large quantities of the expressed protein and that this system is also able to perform many of the post-translational modifications such as glycosylation, folding, phosphorylation and secretion. The receptor cDNA can be inserted into Baculovirus transfer vector (MaxBac 2.0 kit+pBlueBacHis2 Xpress kit, Invitrogen, Carlsbad, Calif.). The recombinant virus can be purified in three rounds and the amount of receptor produced by Sf11 cells in serum-free media can be estimated by Western blot. Additionally, the receptor can be expressed in the COS cell expression system using the pcDNA3.l/His vector (Invitrogen). This is a mammalian expression system in which COS cells can be transfected with receptor cDNA and induced to express protein using a CMV promoter construct. COS cells are easy to transfect using a variety of procedures such as lipofectin.

### Example 4

### Expression and Purification of His-Tagged Recombinant Angiocidin

Recombinant receptor containing six histidine residues linked to the amino terminus was prepared using the Express protein expression system (Invitrogen, Carlsbad, Calif.). Full length cDNA cloned in the PBK-CMV vector was used as a template to generate a PCR product that contained the correct restriction sites enabling the DNA to be ligated into the His tag vector pTrcHISA. This was accomplished by PCR with rTth DNA polymerase, XL (Perkin Elmer, Foster City, Calif.) using the forward primer GGG AGA TCT ATG GTG TTG GM AGC ACT (SEQ ID NO: 12) and the reverse primer GGG GM TTC TCA CTT CTT GTC TTC CTC (SEQ ID NO: 13) containing Bgl II and EcoR1 restriction sites, respectively. The resulting 1.1 kb product contained a Bgl II restriction site at the 5′ end and an EcoR1 site at the 3′ end which was ligated into the vector digested with BamH1 and EcoR1 using T4 DNA ligase.

### Example 5

### Binding of Cys-Ser-Val-Thr-Cys-Gly and TSP-1 to Recombinant Angiocidin

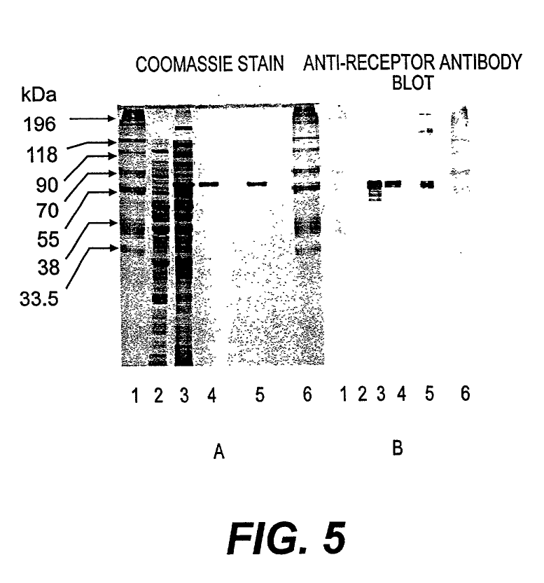

Bacterial lysates containing receptor cDNA inserts and empty vector controls as well as purified His-tag recombinant receptor were analyzed by SDS-PAGE under both reducing and non-reducing conditions. Gels were electroblotted onto nitrocellulose paper and the blots blocked with 1% BSA for 1 hour at room temperature, as shown in FIG. 5.

For Western blotting, membranes were treated with 1:2000 receptor antibody serum in TBS-tween (tris-buffered saline containing 0.05% TWEEN-20™) for 2 hours, washed in TBS-tween, probed for 1 hour with 1:15,000 horseradish peroxidase-conjugated anti-rabbit IgG, washed, and then revealed by ECL (Enhanced Chemiluminescence), Amersham, Arlington Heights, Ill., as shown in FIG. 5.

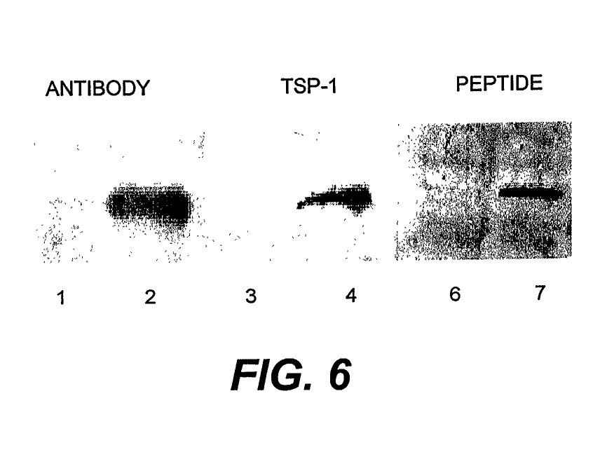

For ligand blotting, membranes were treated with either biotinylated TSP-1 (5 μg/ml) or biotinylated Cys(Acm)-Ser-Val-Thr-Cys(Acm)-Gly (SEQ ID NO: 6) (5 μg/ml) for 1 hour at room temperature, washed in TBS-tween, probed for 1 hour with 1:2000 horseradish peroxidase-avidin, washed, and then revealed by ECL (Enhanced Chemiluminescence), Amersham, Arlington Heights, Ill., as shown in FIG. 6.

Both TSP-1 and Cys(Acm)-Ser-Val-Thr-Cys(Acm)-Gly (SEQ ID NO: 6) were biotinylated using the Pierce protein biotinylation protocol (EZ-Link Sulfo-NHS-LC-Biotin, Pierce Chemical Co Rockfort, Ill.). Unreacted biotin was removed by dialysis.

### Example 6

### Evaluation of Undenatured Angiocidin Binding to TSP-1

Binding of undenatured (in the ligand blot protocol above, the receptor is denatured by SDS) recombinant receptor to TSP-1 was evaluated using the Affinity Sensor System, Cambridge, UK. This is an optical binding method that uses a cuvette to which either ligand or receptor is covalently coupled. A laser beam is used to detect bound proteins to the protein-derivatized cuvette surface. This method is highly sensitive and measures both the association and dissociation rate constants for ligand receptor interactions. The instrument assumes that one molecule of receptor binds one molecule of TSP-1 and calculates the dissociation constant (K_{D}) according to the following relationships:

1) kₐₛₛ[R][TSP-1]=k_{diss}[R-TSP-1] at equilibrium, where kₐₛₛ is the second order rate constant for association and k_{diss} is the first order rate constant for dissociation

2) K_{D}=[R][TSP-1]/[R-TSP-1]=k_{diss}/kₐₛₛ

3) [R-TSP-1]ₜ=[R-TSP-1]_{eq}[1-exp(-kₒₙₜ)], where the instrument response measure in arc seconds is proportional to receptor-TSP-1 complex R-TSP-1].

4) kₒₙ=kₐₛₛ[L]+k_{diss} where kₒₙ is the pseudo-first order rate constant for receptor TSP-1 interaction.

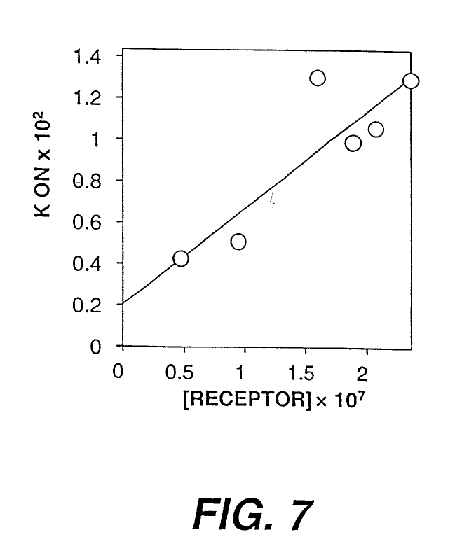

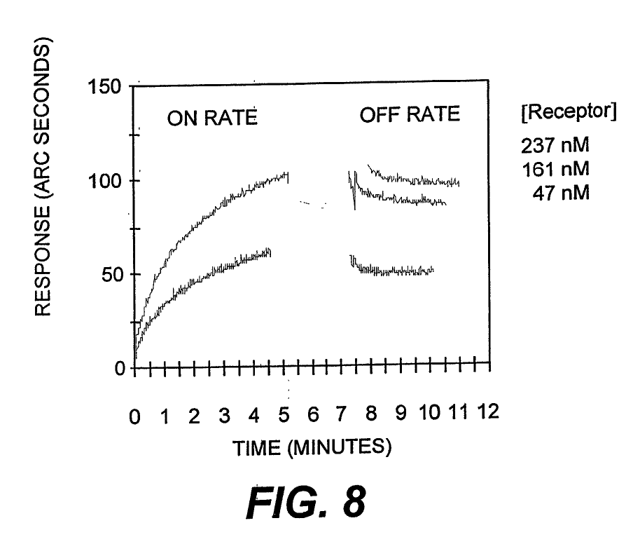

About 1 μg of TSP-1 was coupled to the cuvette through its amino groups to COOH groups on the cuvette surface. Unreacted groups on the cuvette surface were then blocked with ethanolamine and albumin. Receptor at concentrations above 189 nM in HEPES buffered saline, pH 7.00 showed saturable binding after 7 min. and that binding could be partially dissociated with buffer or completely dissociated with low pH buffer. A dissociation constant of 44 nm was calculated from a plot of the pseudo first order rate constant for association versus the concentration of the receptor, as shown in FIG. 7. Instrument response vs time readings shown in FIG. 8, where the instrument response is proportional to the concentration of receptor-TSP-1 complex, were used to plot the data points on FIG. 7.

Addition of the detergent Tween 20 to the buffer did not alter the binding consistent with specific binding. Additionally, extent of receptor binding in the presence of a 10 fold molar excess of Cys(Acm)-Ser-Val-Thr-Cys(Acm)-Gly (SEQ ID NO: 6), a type 1 repeat domain of TSP-1, was 47% of buffer control, whereas a 10 fold molar excess of the scrambled peptide, Val-Cys(Acm)-Thr-Gly-Ser-Cys(Acm) (SEQ ID NO: 7), was 88% of buffer control, suggesting that binding can be partially competed with peptides containing the Cys-Ser-Val-Thr-Cys-Gly (SEQ ID NO: 1) sequence. These results demonstrate cloning of a protein that binds TSP-1.

### Example 7

### Evaluation of Angiocidin and Peptide Binding to Immobilized TSP-1

The methodology set forth in Example 6 was followed except that TSP-1 was immobilized on the cuvette and one of the following solutions was added: receptor alone, peptide plus receptor (peptide:receptor 1000 molar ratio and 100 molar ratio). The peptides used were Val-Cys-His-Ser-Lys-Thr-Arg (SEQ ID NO: 8), Val-Cys(Acm)-His-Ser-Lys-Thr-Arg (SEQ ID NO: 9), and Pro-His-Ser-Arg-Asn (SEQ ID NO: 10). The first two peptides are derived from the binding portion of the receptor, where it interacts with the Cys-Ser-Val-Thr-Cys-Gly (SEQ ID NO: 1) portion of the TSP-1 protein. The third peptide is a control.

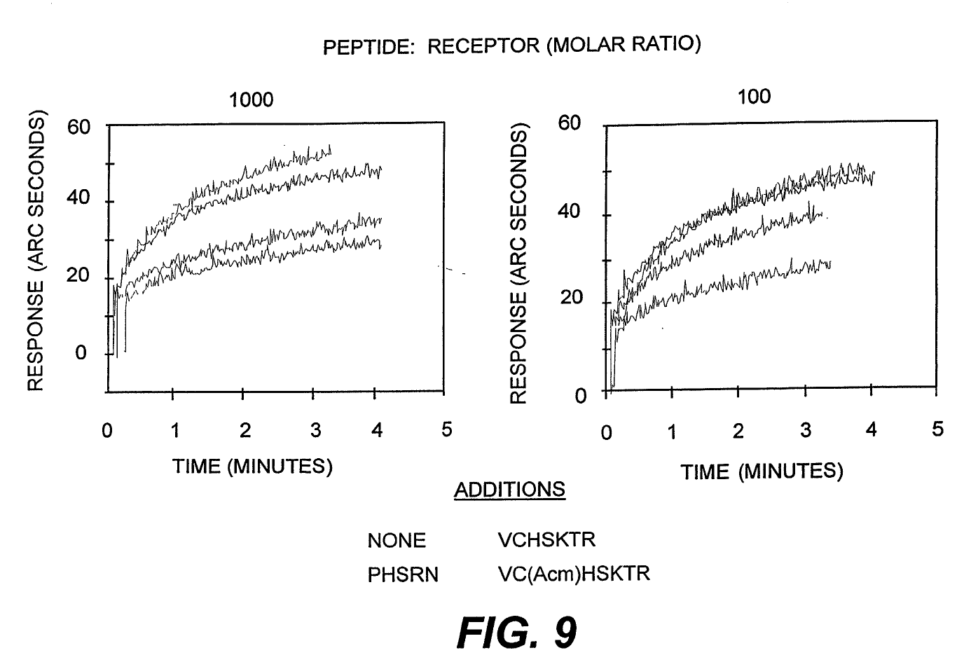

FIG. 9 shows that the peptide Val-Cys-His-Ser-Lys-Thr-Arg (SEQ ID NO: 8) inhibits binding of the receptor with the immobilized TSP-1, by binding to the TSP and competitively inhibiting binding of the receptor. This interaction is correlated with concentration, as seen by comparing the different molar ratios of peptide to receptor.

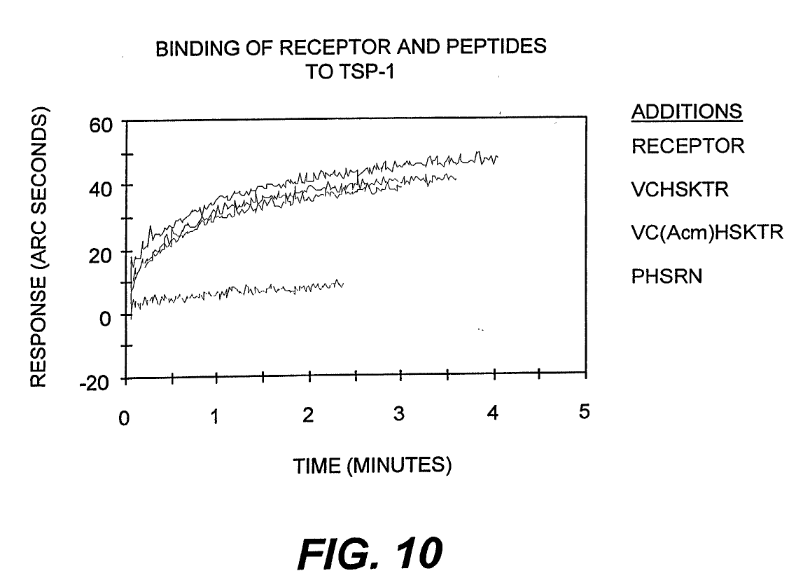

Additionally, FIG. 10 shows the direct binding of the receptor-derived peptides to the TSP-1 immobilized in the cuvette. With the receptor as a positive control and Pro-His-Ser-Arg-Asn (SEQ ID NO: 10) as a negative control, it can be seen that the peptides Val-Cys-His-Ser-Lys-Thr-Arg (SEQ ID NO: 8) and Val-Cys(Acm)-His-Ser-Lys-Thr-Arg (SEQ ID NO: 9) bind directly to the immobilized TSP-1.

These figures show that the Val-Cys-His-Ser-Lys-Thr-Arg (SEQ ID NO: 8) region on the receptor of the present invention binds to the TSP-1 protein.

### Example 8

### Evaluation of Angiocidin Binding to Immobilized TSP-1 and C(Acm)SVTC(Acm)G (SEQ ID NO: 6)

The methodology set forth in Example 6 was followed except that TSP-1 and Cys(Acm)-Ser-Val-Thr-Cys(Acm)-Gly (SEQ ID NO: 6) were immobilized on cuvettes and the receptor was added to the cuvettes. The Acm version of the peptide was used to increase its stability in the experiment.

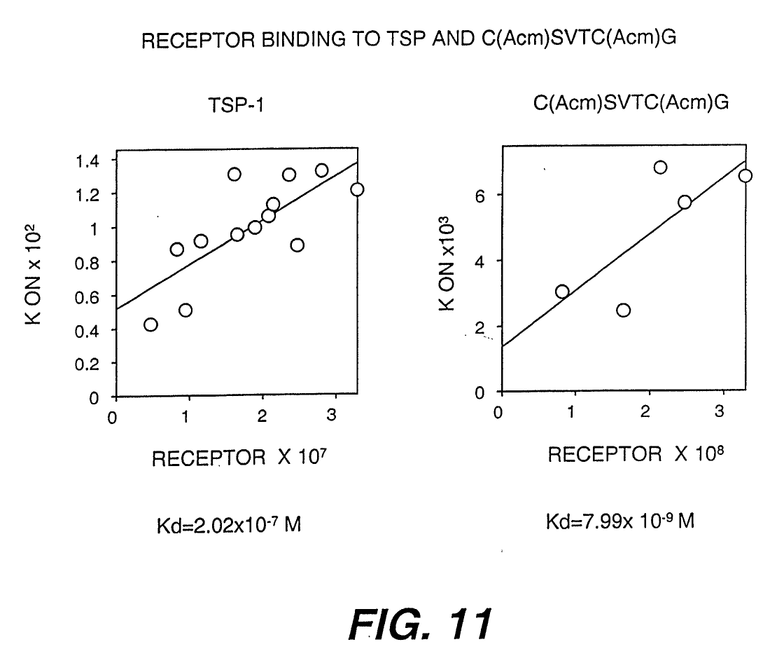

FIG. 11 shows that both TSP-1 and the peptide bind to the receptor. This demonstrates that the Cys-Ser-Val-Thr-Cys-Gly (SEQ ID NO: 1) region of TSP-1 binds to the receptor.

### Example 9

### Surface Labeling of Angiocidin

Intact, growing A549 lung carcinoma cells were surface labeled with ¹²⁵I-Iodine using lactoperoxidase as described by Tuszynski et al., Anal. BioChem. 106:118-122 (1980). Briefly, a 75 mm flask containing a near confluent monolayer of cells was rinsed three times with 10 ml of DMEM. Then the cell layer was covered with 5 ml of DMEM containing 0.2 units/ml lactoperoxidase and 500 μCi of ¹²⁵I-Iodine. Five one μl aliquots of 30% H₂O₂ were added with gentle mixing at one minute intervals. The reaction was then stopped by the addition of 5 μl of a 1 mM NaN₃, the monolayer washed three times with DMEM, and cells harvested for purification of Cys-Ser-Val-Thr-Cys-Gly (SEQ ID NO: 1) binding proteins.

Analysis of the labeled material by SDS-gel electrophoresis followed by autoradiography revealed that the Mw=50,000 polypeptide under non-reduced conditions labeled by in vitro iodination was labeled (FIG. 4, lane 5).

The receptor bound TSP-1 in a time-dependent manner which became time-independent after 60 min. The binding was maximal in the presence of both 1 mM CaCl₂ and 1 mM MgCl₂ and whereas a small but significant amount of binding occurred in the presence of 1 mM EDTA. This example shows not only that the receptor and the TSP-1 bind in a time-dependent manner, but also that the receptor is expressed on the surface of the cell.

### Example 10

### Immunohistochemistry of Angiocidin

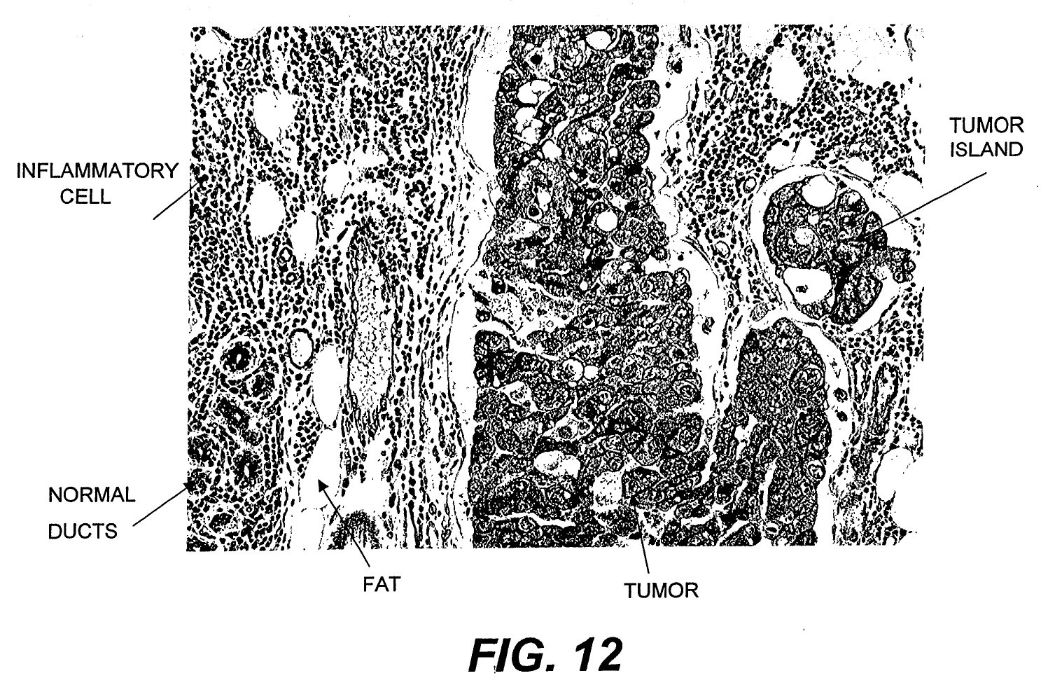

FIG. 12 demonstrates the localization of the receptor in breast tumors. The tumor is located in a large vertical stripe in the center of the figure, with two islands on the right hand side of the figure. The smaller cells located to the right and left are inflammatory cells, and the large white cells are fat tissue. For comparison a cluster of normal breast ducts are shown in the lower left hand corner of the figure.

The tissue was fixed in cold 95% ethyl alcohol for 10 minutes and paraffin embedded. Sections (5 μm) were cut and mounted on glass microscope slides. Slides were deparaffinized and rehydrated by sequential incubation in graded xylene-ethanol solutions. Endogenous peroxidase activity was quenched by treatment with 3% H₂O for minutes, followed by water wash. Slides were then washed in phosphate buffered saline (PBS) and treated with a 5-20 μg/ml solution of primary IgG (either immune or nonimmune IgG) in PBS containing 0.1% BSA (PBS-BSA) for 30 minutes. After washing in PBS-BSA, slides were treated with a 1:250 dilution of the secondary biotinylated antibody for 30 minutes, washed, and developed according to the procedure provided by the Vectastain ABC Immunoperoxidase Staining Kit, Vector Laboratories (Burlingame, Calif.). Slides were then counterstained with hematoxylin, mounted with coverslips, and examined by bright field microscopy.

The stained receptor can be visualized around the border of the tumor cells, but not around the normal cells in the lower left hand corner. This shows that the receptor is associated with the cell membrane, and that it is more concentrated in the tumor cells.

### Example 11

### Transient Transfection and Cell Adhesion Assay

Bovine Aorta Endothelial Cells (BAEC) and MDA-MB-231 cells, breast carcinoma cells, were transfected with purified DNA encoding for the receptor by the Wizard Plus Kit (Promega, Wis.). The DNA is incorporated into the cells using the Superfect transfection reagent (Qiagen, Calif.). Cells were plated in 6 well plates and upon 80% confluency transfection is performed. 12 μl of the reagent was used as well as 2.5 μg of the DNA, with minimal concentration of 0.1 μg/pl. Superfect-DNA complex formation was performed in a serum free and antibiotic free medium. Cells were incubated at 37° C. for 3-4 hours. Then media was changed and 48 hours post transfection they were harvested for the adhesion assays.

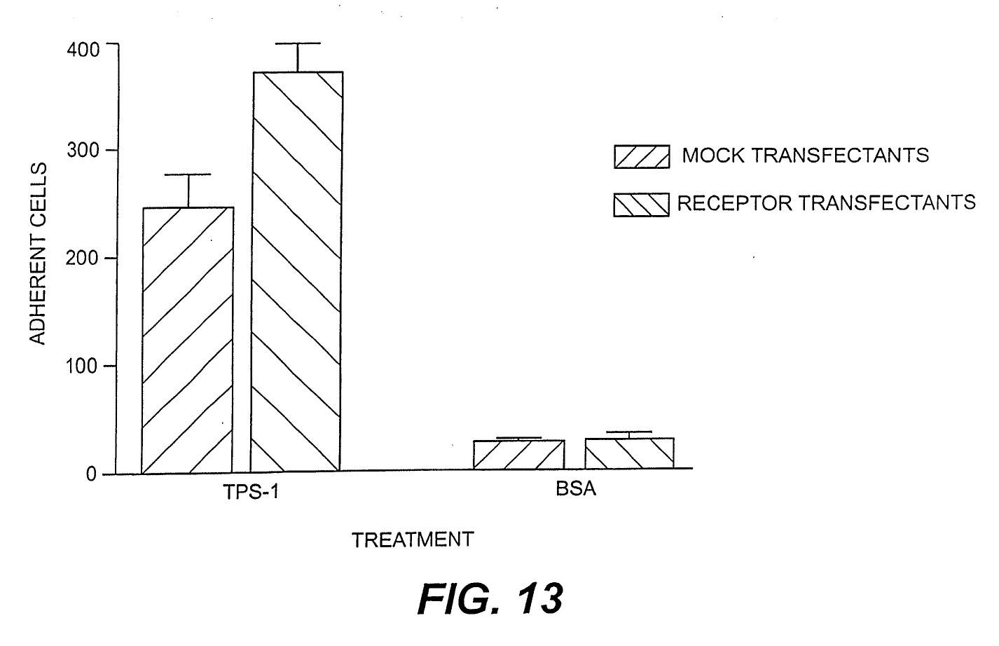

For the adhesion assay, in a 96 well plate, duplicate wells were covered with either TSP-1 (40 μg/ml), fibronectin (40 μg/ml), or and 1% bovine serum albumin (BSA). The wells were dried out overnight and then blocked with BSA. 100 μl of a suspension containing 2×10⁵ cells were plated in the protein covered wells and incubated at 37^{□} C. for 20 minutes to 1 hour. The non-adherent cells were removed and the wells were washed with a Hepes buffer. The adherent cells were fixed with 2.5% glutaraldehyde for 10 minutes and stained with 0.2% Giemsa. The stain was washed off and the cells were counted in a field of 1 mm square. Cells adhering to BSA were considered background while cells adhering to fibronectin were the positive control. These data are displayed in FIG. 13.

### Example 12

### Transient Transfection and Cell Adhesion Assay

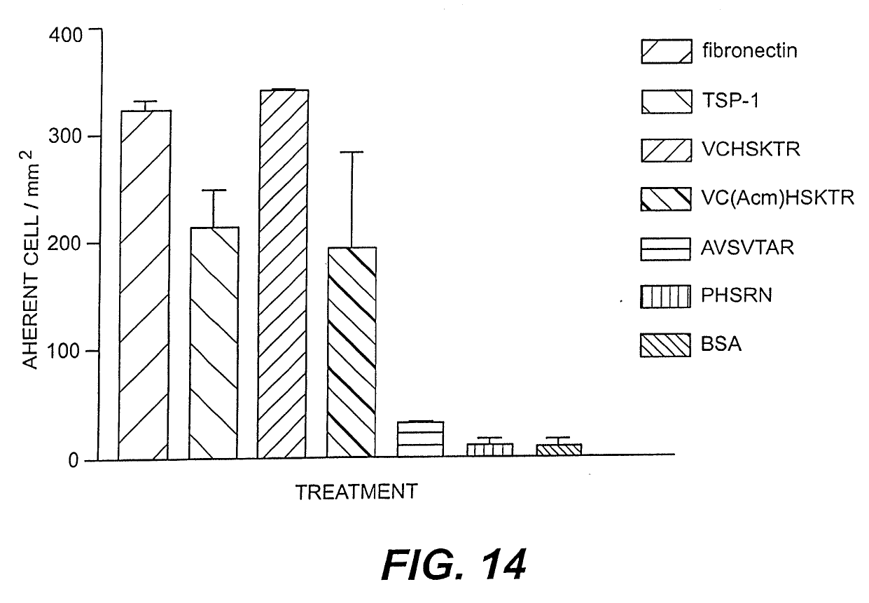

The method of Example 12 was followed except the receptor peptides Val-Cys-His-Ser-Lys-Thr-Arg (SEQ ID NO: 8) and Val-Cys(Acm)-His-Ser-Lys-Thr-Arg (SEQ ID NO: 11) were immobilized on the plates. TSP-1 and fibronectin were also immobilized on plates, as well as negative control peptides (Ala-Ser-Val-Thr-Ala-Arg (SEQ ID NO: 11) and Pro-His-Ser-Arg-Asn (SEQ ID NO: 10)) and bovine serum albumin. The results of this experiment, FIG. 14, show that the receptor peptides cause the cells to adhere to the plates, with similar affinity to the positive controls fibronectin and TSP-1. This provides support for the theory that another protein may be associated with TSP-1 and its receptor, or that the receptor is released and rebound to the membrane of the cell by another protein.

### Example 13

### Transient Transfection and Cell Adhesion Assay

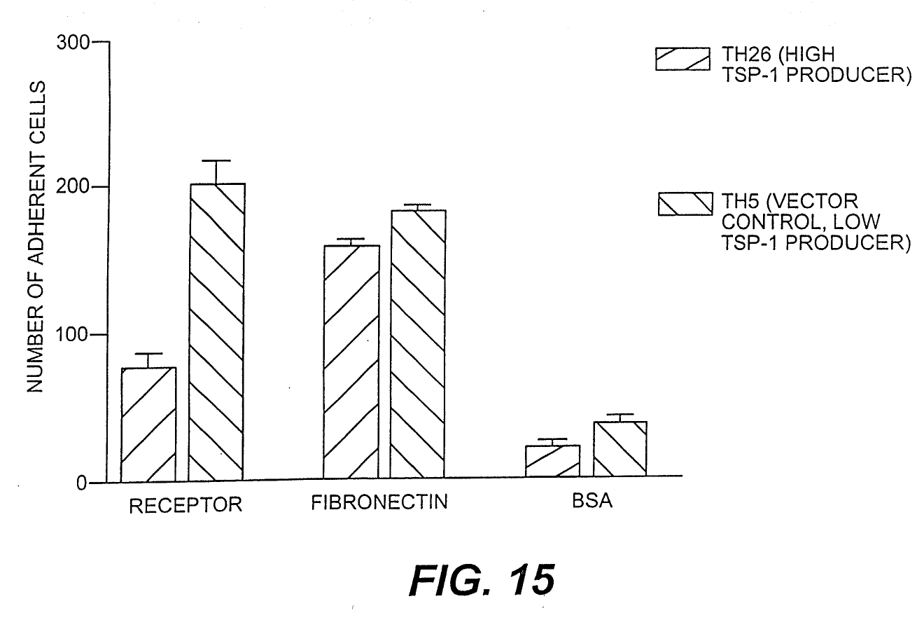

The method of Example 12 was followed except the whole receptor protein was immobilized on the plates, and cells transfected with either TSP-1 cDNA or a vector control were applied to the plates. The cells, which naturally express a low level of TSP-1, were transfected to over express the protein. FIG. 15 shows that the cell transfected with TSP-1 cDNA bound more to the plates with receptor protein than the control cell line (2.5 times better, p<0.001). Fibronectin and BSA were used as positive and negative controls, respectively, for cell adhesion. This evidence bolsters the theory that the receptor of the present invention binds to thrombospondin.

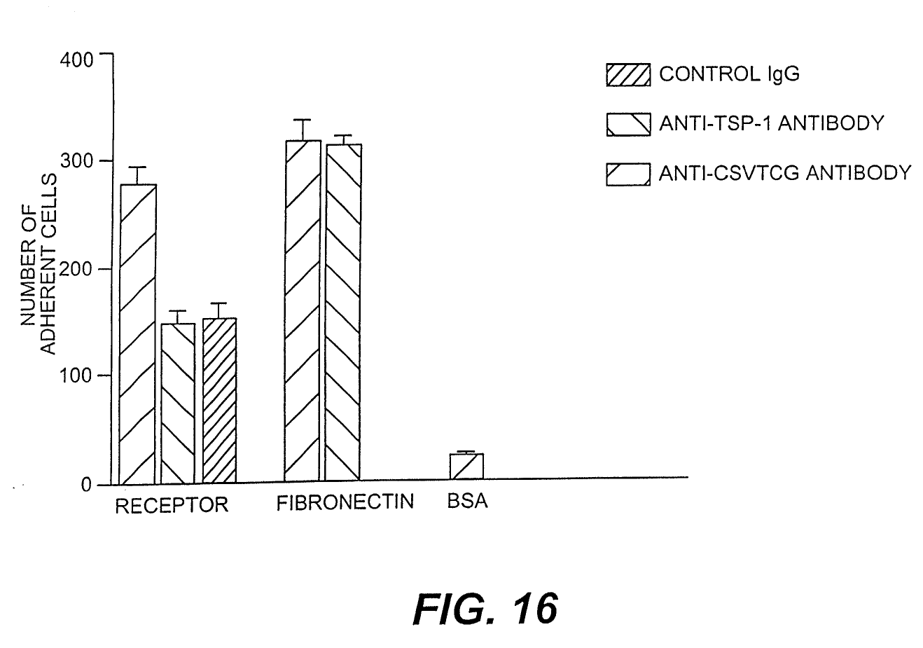

This specific interaction was confirmed by adding anti-TSP-1 antibodies, Anti-Cys-Ser-Val-Cys-Thr-Gly (SEQ ID NO: 1), and control IgG to the system. FIG. 16 shows that both the anti-TSP-1 and the anti-Cys-Ser-Val-Cys-Thr-Gly (SEQ ID NO: 1) antibodies inhibited adhesion of TSP-1 expressing cells to the receptor bound to the plate.

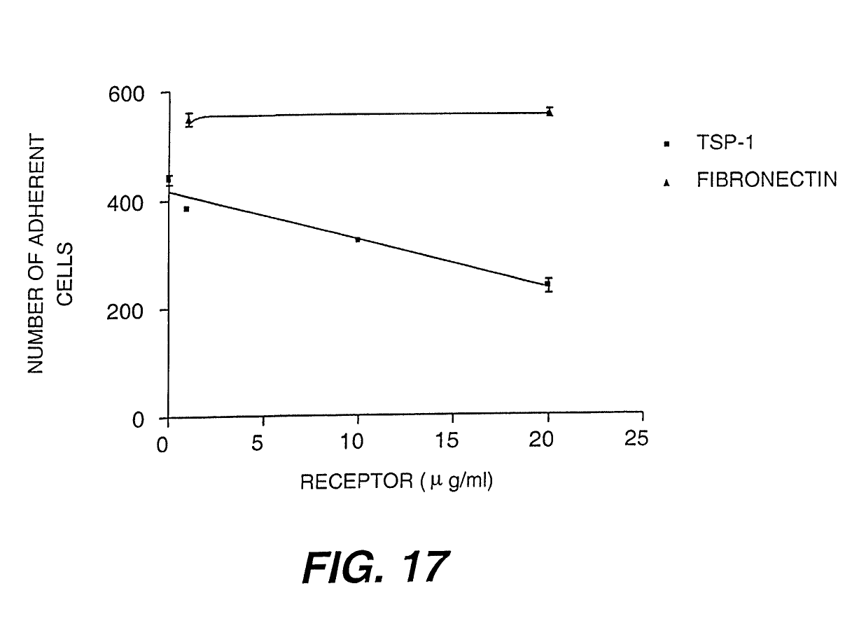

Furthermore, addition of unbound receptor in a solution to the adhesion system reduced the adhesion of the cells to the plate. FIG. 17 shows that the receptor itself competitively inhibits the adhesion of the nontransfected, naturally TSP-1 expressing cells to the receptor bound to the plate, helping to show that this is the interaction causing the adhesion.

### Example 14

### Production of Antibodies to Angiocidin, the TSP-1 Receptor

Either native or synthetic (recombinant) Cys-Ser-Val-Thr-Cys-Gly (SEQ ID NO: 1)-specific receptor protein can be used to produce antibodies, both polyclonal and monoclonal. If polyclonal antibodies are desired, purified receptor protein is used to immunize a selected mammal (e.g., mouse, rabbit, goat, horse, etc.) and serum from the immunized animal later collected and treated according to known procedures. Compositions containing polyclonal antibodies to a variety of antigens in addition to the receptor protein can be made substantially free of antibodies which are not anti-receptor protein antibodies by passing the composition through a column to which receptor has been bound. After washing, polyclonal antibodies to the receptor are eluted from the column. Monoclonal anti-receptor protein antibodies can also be readily produced by one skilled in the art. The general methodology for making monoclonal antibodies by hybridomas is well known. Immortal, antibody-producing cell lines can also be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies and T-Cell Hybridomas” (1981); Kennett et al., “Monoclonal Antibodies” (1980).

By employing TSP-1 receptor protein (native or synthetic) as an antigen in the immunization of the source of the B-cells immortalized for the production of monoclonal antibodies, a panel of monoclonal antibodies recognizing epitopes at different sites on the receptor protein molecule can be obtained. Antibodies which recognize an epitope in the binding region of the receptor protein can be readily identified in competition assays between antibodies and TSP-1. Such antibodies could have therapeutic potential if they are able to block the binding of TSP-1 to its receptor in vivo without stimulating the physiological response associated with TSP-1 peptide binding.

Specifically, polyclonal Cys-Ser-Val-Thr-Cys-Gly (SEQ ID NO: 1)-specific receptor antiserum was raised in a rabbit by standard procedures after four 50 μg injections every three to four weeks. The first injection was given with complete Freund's adjuvant and subsequent injections were administered with incomplete Freund's adjuvant. Antibody titers and specificity were determined by ELISA. Native purified receptor was used in this Example.

ELISA assays were performed following standard procedures. Briefly, microtiter plates were coated with 2 μg of Cys-Ser-Val-Thr-Cys-Gly (SEQ ID NO: 1)-specific receptor, fibronectin or BSA and blocked with 1% BSA for 1 hour. Wells were incubated for 1 hr with 50 μl of various dilutions of the first antibody in 10 mM phosphate buffer, pH 7.4, containing 150 mM NaCl and 0.05% Tween-20 (PBS-T). Wells were then washed three times in PBS-T and incubated for 1 hr with 50 μl of a 1:800 dilution in PBS-T of alkaline phosphatase coupled rabbit anti-goat IgG. Wells were washed three times with PBS-T followed with three washes of PBS-T buffer containing no TWEEN-20™ and treated with 50 μl of alkaline phosphatase substrate solution (1 mg/ml of p-nitrophenylphosphate in 0.10M glycine, pH 10.4, containing 1 mM ZnCl₂ and 1 mM MgCl₂). After 30 minutes, color development was stopped by the addition of 5 μl of 1 N NaOH and absorbances determined at 405 nm.

The antibody was monospecific as determined by direct ELISA as shown in

Table 1.

TABLE 1
Monospecificity of the Angiocidin
Antibody Absorbance (405 nm)

| | | | Cys-Ser-Val- |
|---|---|---|---|
| | | | Thr-Cys-Gly |
| | | | (SEQ ID NO: 1)- |
| | | | Specific |
| | BSA | Fibronectin | Receptor |
| Preimmune | 0.123 +/− | 0.135 +/− | 0.130 +/− |
| Serum | 0.005 | 0.006 | 0.007 |
| Anti-Cys-Ser- | 0.134 +/− | 0.176 +/− | 0.665 +/− |
| Val-Thr- | 0.007 | 0.004 | 0.003 |
| Cys-Gly | | | |
| (SEQ ID NO: 1) | | | |
| Specific | | | |
| Receptor | | | |

### Example 15

### Adhesion Inhibition by Antibody

The following experiment was performed to determine the ability of the anti-Cys-Ser-Val-Thr-Cys-Gly (SEQ ID NO: 1)-specific receptor antibody to inhibit adhesion of cancer cells to TSP-1. The A549 lung carcinoma expresses the thrombospondin receptor protein. Detachable microtiter wells (Immulon 4 Removawell) were coated overnight at 4° C. with either 50 μl of a 40 μg/ml TSP-1, fibronectin, or laminin solution in 20 mM bis-tris-propane buffer, pH 6.5 and blocked for one hour with 200 μl of 1% BSA. A549 cells and 200 μg/ml of IgG for anti-Cys-Ser-Val-Thr-Cys-Gly (SEQ ID NO: 1)-specific receptor or non-immune antisera were incubated for 30 minutes and centrifuged to remove unbound antibody. The pellet was resuspended in DMEM and the cells incubated in the protein-coated wells for 60 minutes at 37° C. The number of cells adhering to the microtiter well surface was counted. The results in Table 2 are presented as % of non-immune IgG-treated adherent cells. Table 2 shows that anti-Cys-Ser-Val-Thr-Cys-Gly (SEQ ID NO: 1)-specific receptor antibody inhibits A549 cell adhesion to TSP-1-coated surfaces, but had no effect on cell adhesion to fibronectin or laminin. The antibody also inhibited adhesion of TSP-1 to the tissue culture plastic.

TABLE 2
Adhesion Inhibition by Antibody

| Protein Substrate | % Adhering Cells |
|---|---|
| Thrombospondin | 10.5% |
| Fibronectin | 101% |
| Laminin | 103% |

### Example 16

### Effect of Angiocidin on Angiogenesis

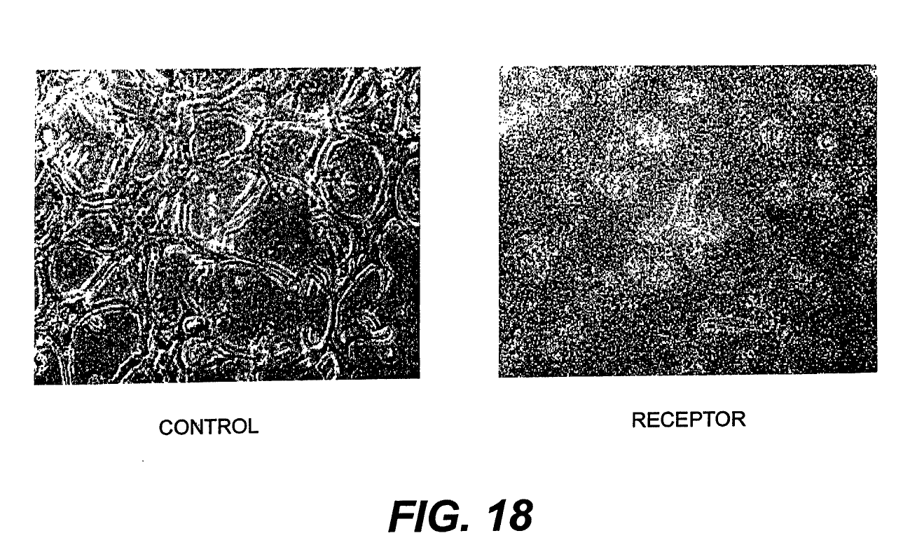

An experiment was performed to evaluate the effect of angiocidin on angiogenesis. Bovine aortic endothelial cells (BAEC) were plated on a collagen matrix. Next, the cells were over-layered with collagen. Angiocidin (37 μg/ml) was added on top of the cells in the treatment plate, and the control plate only received buffer. After 24 hours, phase contrast photomicrographs (200×) were taken. The results are shown in FIG. 18. In the control plate, the BAEC cells rearranged themselves into a network of microvessels. In the angiocidin-treated plate, however, the microvessels did not form and the cells appeared dead.

This collagen assay is a well recognized model for angiogenesis. Qian et al., Thrombospondin-1 modulates angiogenesis in vitro by up-regulation of matrix metalloproteinase-9 in endothelial cells, Exp. Cell Res. 235:403-412 (1997). These results demonstrate that angiocidin is an effective inhibitor of angiogenesis.

### Example 17

### Effect of Angiocidin on Microvessel Stability

The experiment in this example was performed as in Example 16, however, no treatment was given to the cells initially. After 24 hours, microvessels formed in both samples, and looked similar to the control plate in FIG. 19. Buffer and angiocidin were then added to the control and treatment plates, respectively. After an additional 24 hours, Hoffman interference photomicrographs were taken. Here, the control was not affected. However, the addition of angiocidin disrupted the microvessels that had already formed in the treatment plate. Results are shown in FIG. 19. This demonstrates that angiocidin not only prevents angiogenesis, but also reverses the formation of vessels.

### Example 18

### Effect of Angiocidin on Morphology of Bovine Aortic Endothelial Cells

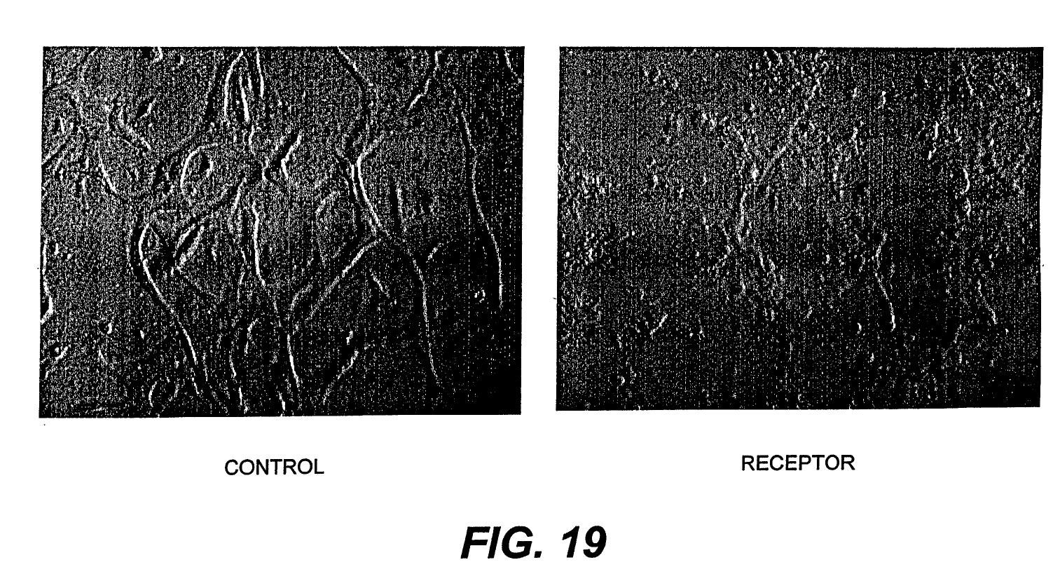

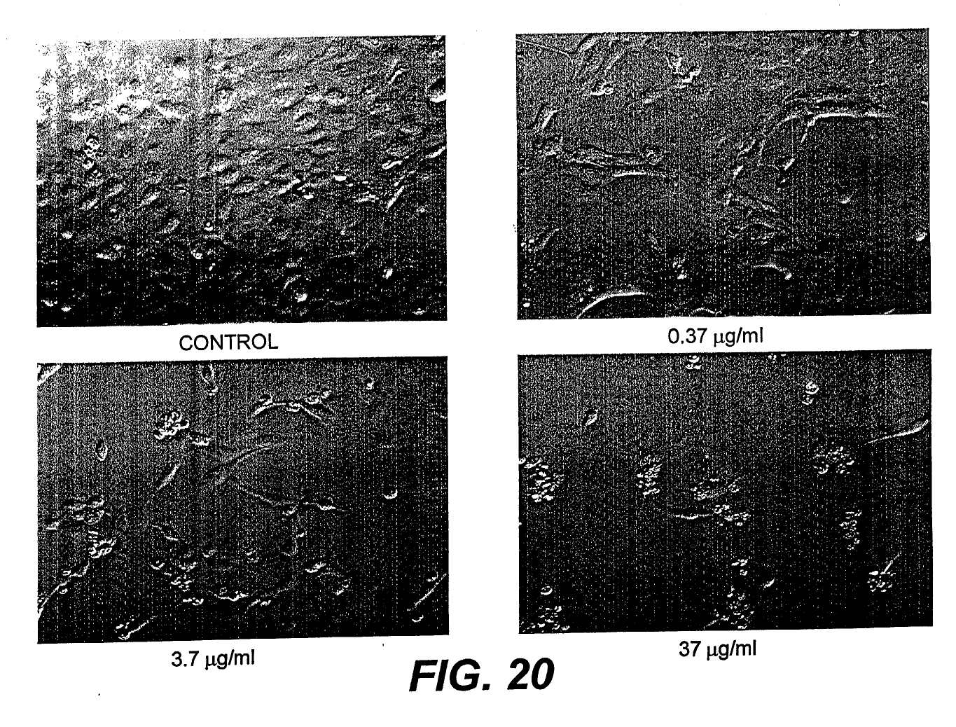

In this experiment, BAEC cells in monolayer cultures were plated for 24 hours in serum-free medium containing 1% BSA in the presence of increasing concentrations of angiocidin (control=none, 0.37 μg/ml, 3.7 μg/ml, 37 μg/ml). Hoffman interference microscopy (100×) was used to photograph the cells. With increasing concentrations of angiocidin, the BAEC cells elongated, detached from the plate, aggregated, and died. Results are shown in FIG. 20.

### Example 19

### Effect of Angiocidin on Cell Viability

Bovine aortic endothelial cells (BAEC), human umbilical vein endothelial cells (HUVEC), fibroblast cells, A549 human lung carcinoma cells (A549), MDA-MB231 human breast carcinoma cells (MB231), MCF7 human breast carcinoma cells (MCF7) were treated with 37 μg/ml of receptor, or buffer alone, for 24 hours. Viability of the cells was measured using the ALAMAR BLUE™ assay, which measures the capacity of cells to metabolize the ALAMAR BLUE™ dye. The ALAMAR BLUE™ assay (available from Biosource International, Camarillo, Calif.) quantitatively measures the proliferation of cell lines and can establish the relative cytotoxicity of chemical agents. The assay incorporates a fluorometric/colorimetric growth indicator based on detection of metabolic activity. The system incorporates an oxidation-reduction (redox) indicator that both fluoresces and changes color in response to chemical reduction of growth medium resulting from cell growth. This causes the redox indicator to change from its oxidized, non-fluorescent, blue form to its reduced, fluorescent, red form. Data can be collected using either fluorescence-based instrumentation (530-560 nm excitation wavelength and 590 nm emission wavelength) or absorbance-based instrumentation (570 nm and 600 nm).

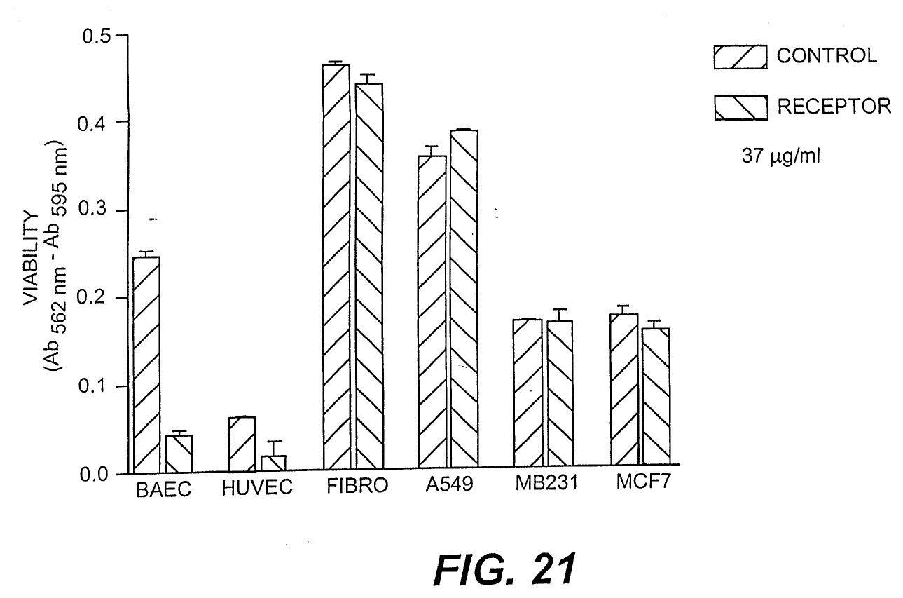

BAEC and HUVEC cell lines have decreased viability in the presence of the receptor, suggesting that TSP is a requirement for viability in these cell lines, as shown in FIG. 21. Endothelial cell viability is decreased by 70-80% after treatment with angiocidin. No significant difference was seen in the fibroblast, A549, MB231, and MCF7 cell lines, suggesting that TSP is not a requirement for viability in for these cells.

### Example 20

### Effect of Angiocidin on Viability of Bovine Aortic Endothelial Cells (BAEC) and Bovine Smooth Muscle Cells (BSM)

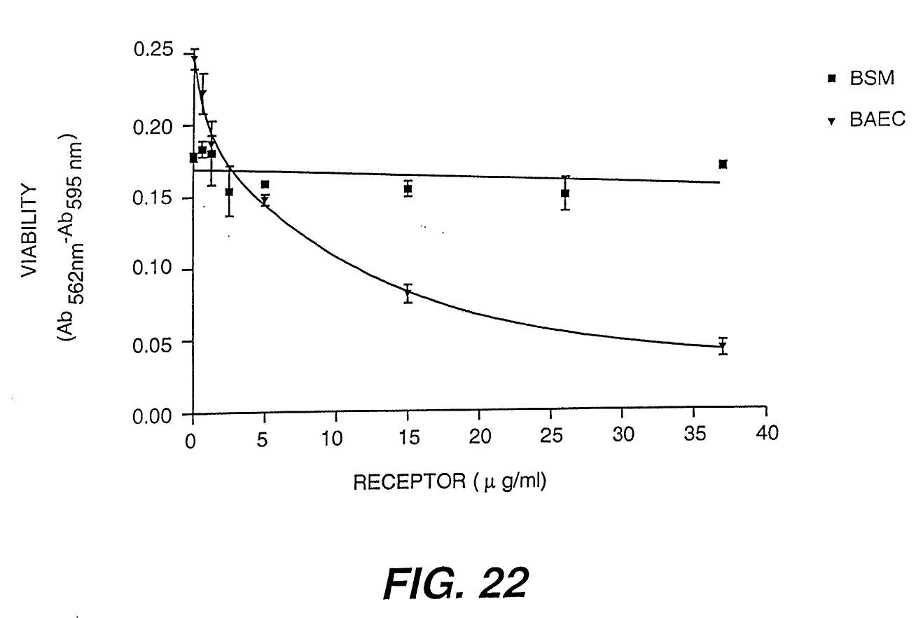

BAEC and BSM cells were treated with increasing concentrations of angiocidin (0, 0.625, 1.25, 2.5, 5, 15, 26 and 37 μg/ml) for 24 hours. Cell viability was measured using the ALAMAR BLUE Tm assay. Angiocidin has a dose dependent inhibition of BAEC cell viability, demonstrating a first order, single constant, exponential decay curve, as shown in FIG. 22. In contrast, BSM cells are unaffected.

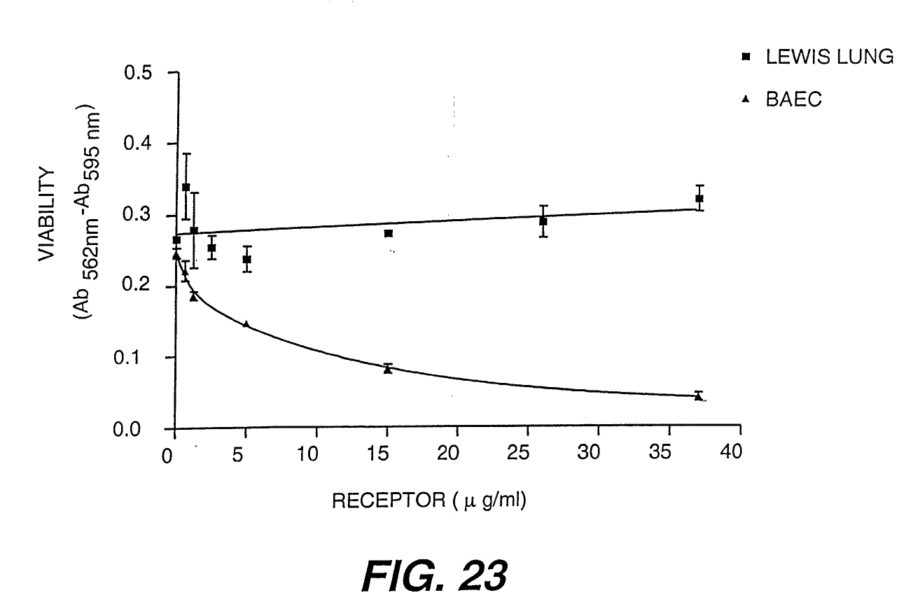

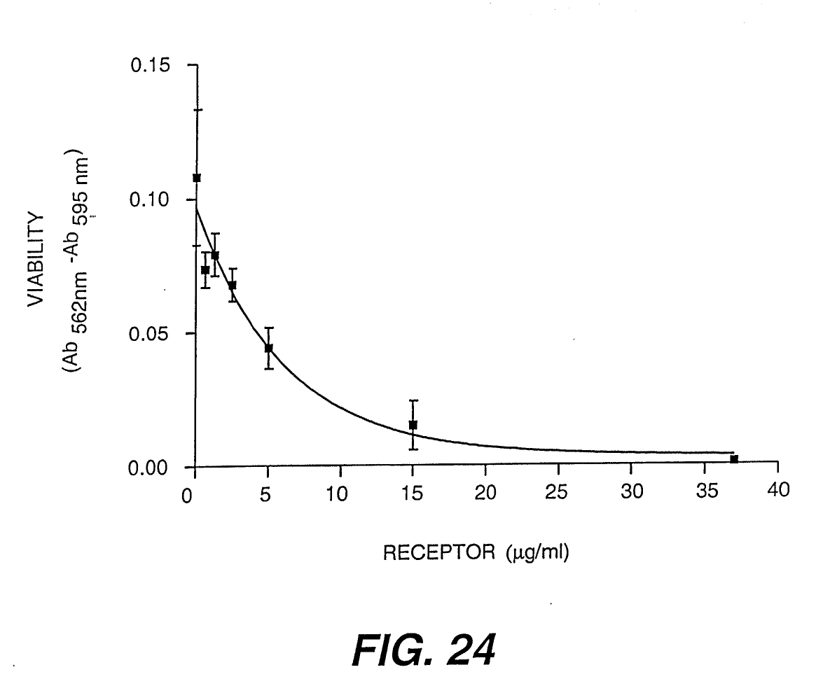

Similarly, the effect of receptor on viability of BAEC cells was compared to mouse Lewis lung carcinoma cells, using the same method. Angiocidin decreases viability of BAEC cells, but does not affect the Lewis lung cells, as shown in FIG. 23. This demonstrates that angiocidin does not directly affect the viability of the Lewis lung cells. The same experiment was performed for HUVEC cells, decreasing their viability. The results are shown in FIG. 24.

### Example 21

### Effect of Angiocidin on Viability of Human Umbilical Vein Endothelial Cells

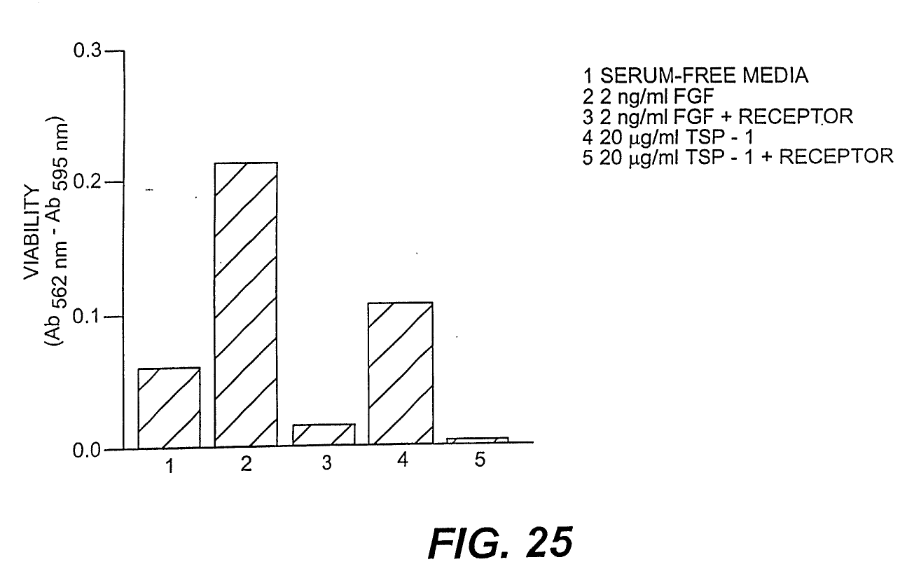

The effect of angiocidin on HUVEC cell viability was evaluated, and FGF and TSP-1 were added to determine whether they ameliorated the angiocidin effect on cell viability. FGF (Fibroblast Growth Factor) is an endothelial cell mitogen, which promotes cell growth. Both FGF (2 ng/ml) and TSP-1 (20 μg/ml) alone stimulated cell growth above control. However, neither the addition of FGF or TSP-1 reversed the inhibition of angiocidin (37 μg/ml). Results are presented in FIG. 25. TSP-1 was expected to reverse the inhibition of angiocidin; however, quantities added may have been insufficient to provide the correct molar ratio.

### Example 22

### Receptor-Mediated Viability of Bovine Aortic Endothelial Cells

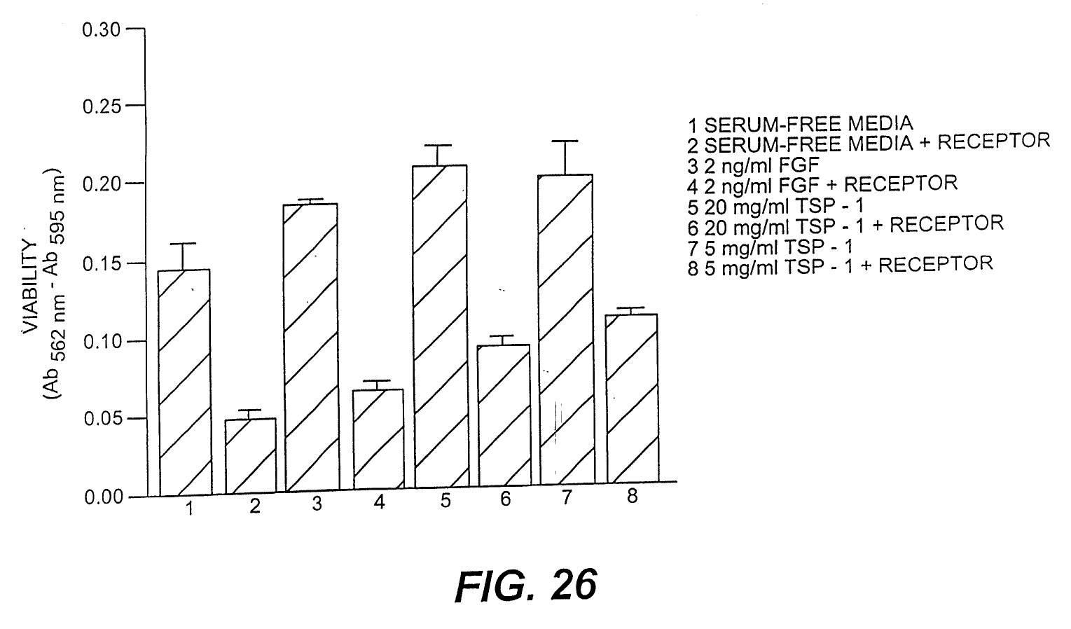

The methods of Example 21 were followed, except BAEC cells were used. Additionally, TSP-1 was added at both 20 μg/ml and 5 μg/ml. These results, as shown in FIG. 26, illustrate that TSP can ameliorate some of the inhibition of angiocidin compared to control.

### Example 23

### Receptor Binding Assay

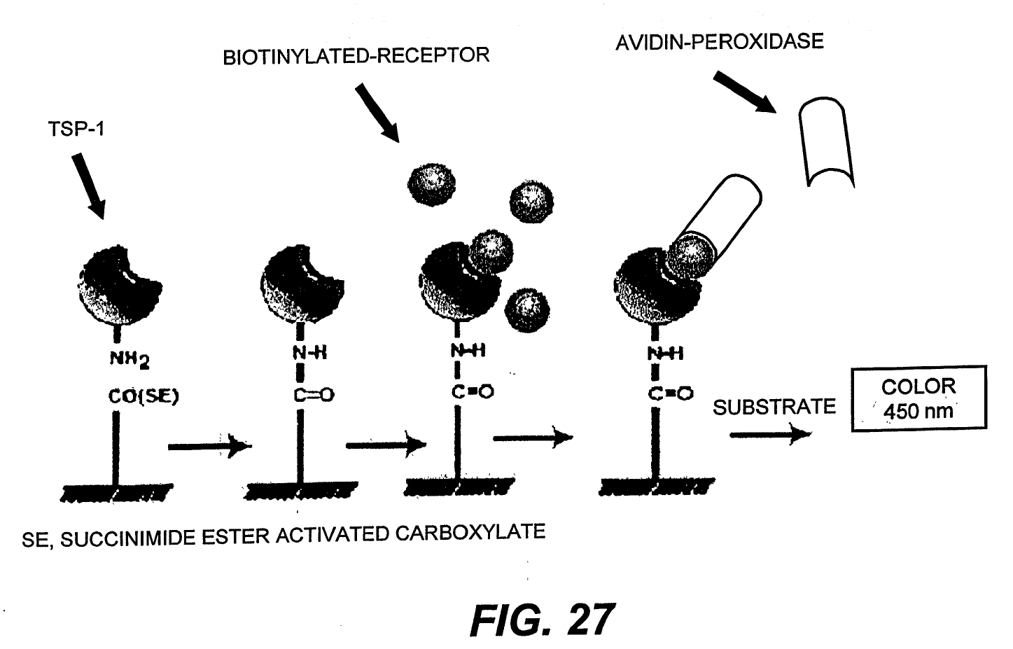

Aschematic for the receptor binding assay is shown in FIG. 27. In the following experiments, TSP-1 was covalently bound to a substrate, biotinylated angiocidin was added to the plate, and avidin-peroxidase was added to measure how much biotinylated angiocidin was attached to the TSP-1. The avidin-peroxidase was measured using a spectrophotometer at an absorbance of 450 nm.

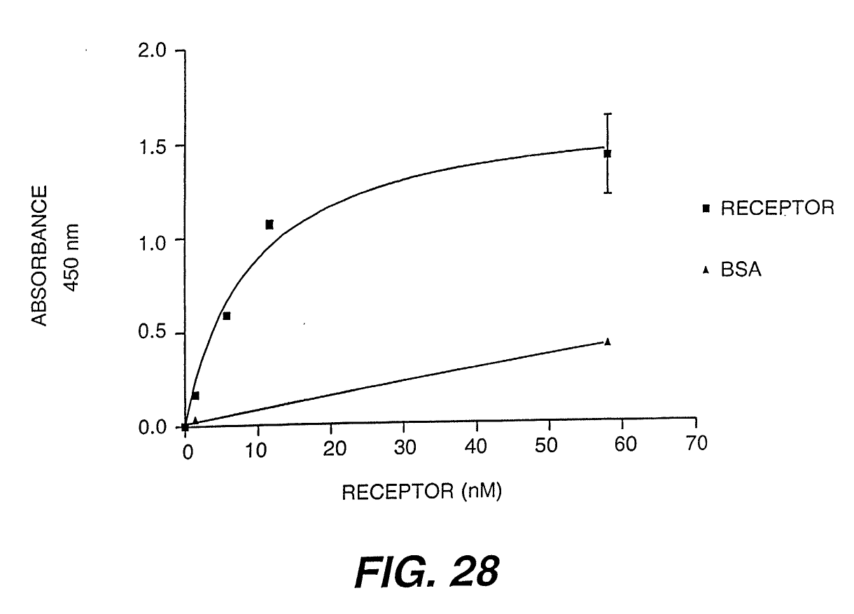

The binding of angiocidin to immobilized TSP-1 is shown in FIG. 28. The binding shows saturable binding with a K_{D}=9 nM. BSA was used as a negative control.

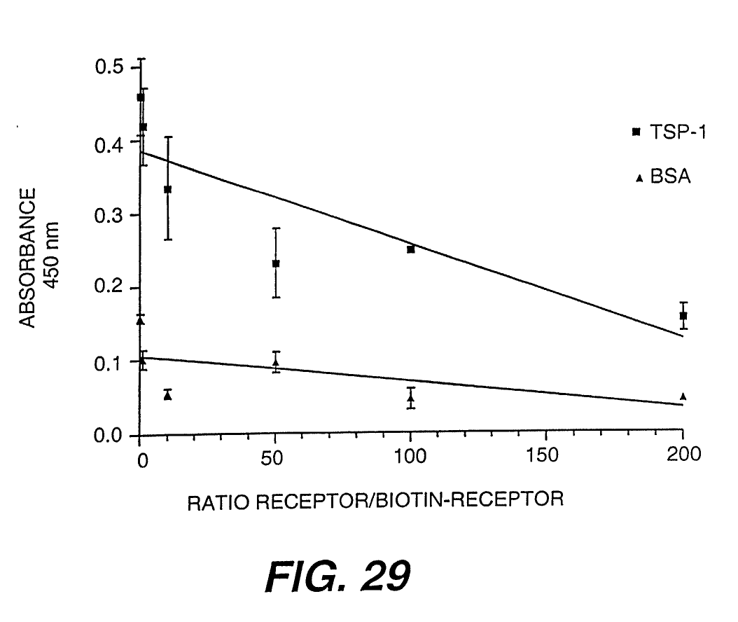

Free angiocidin was added to the system to compete with the biotinylated angiocid in. FIG. 29 shows the competition effect of angiocidin on binding of the biotin-angiocidin complex to TSP-1. Immobilized BSA was used as a negative control. With an increasing ratio of angiocidin to biotin-angiocidin complex, the binding decreased linearly.

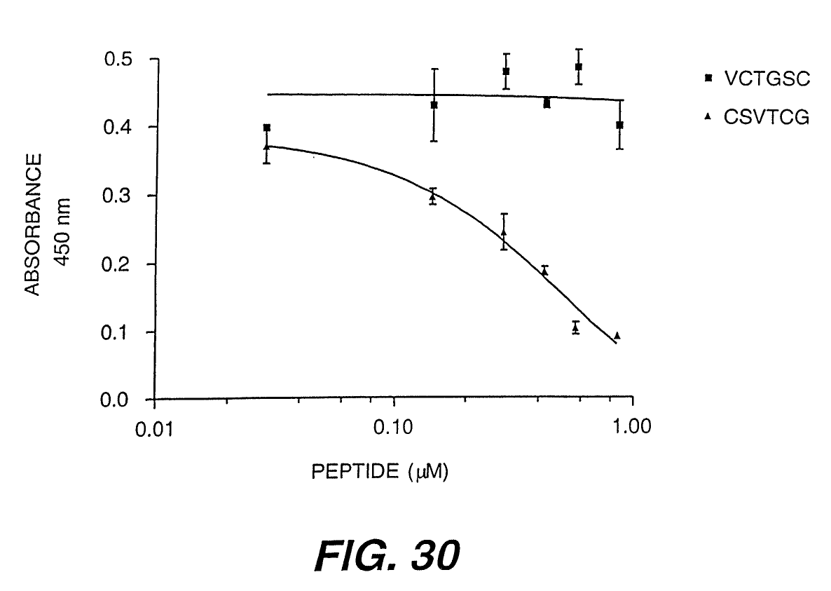

The TSP-1 peptide Cys-Ser-Val-Thr-Cys-Gly (SEQ ID NO: 1) was added to the system to compete with the TSP-1 on the plate for binding with the biotinylated angiocidin. Cys-Ser-Val-Thr-Cys-Gly (SEQ ID NO: 1) effectively competed with TSP-1 for the biotin-angiocidin complex, as shown in FIG. 30. The scrambled peptide Val-Cys-Thr-Gly-Ser-Cys (SEQ ID NO: 15) was used as a negative control and had no effect.

### Example 24

### Identification of Angiocidin Binding Peptides

The phage display peptide library kit, from New England Biolabs (Beverly, Mass.), was used to identify peptides that bind to angiocidin. A library of phage-displayed peptides was incubated with a plate (or bead) coated with the target receptor, the unbound phage was washed away, and the specifically-bound phage was eluted. The eluted phage was then amplified and taken through additional cycles of biopanning and amplification to successively enrich the pool of phage in favor of the tightest binding sequences. After 3 rounds, individual clones were characterized by DNA sequencing and ELISA.

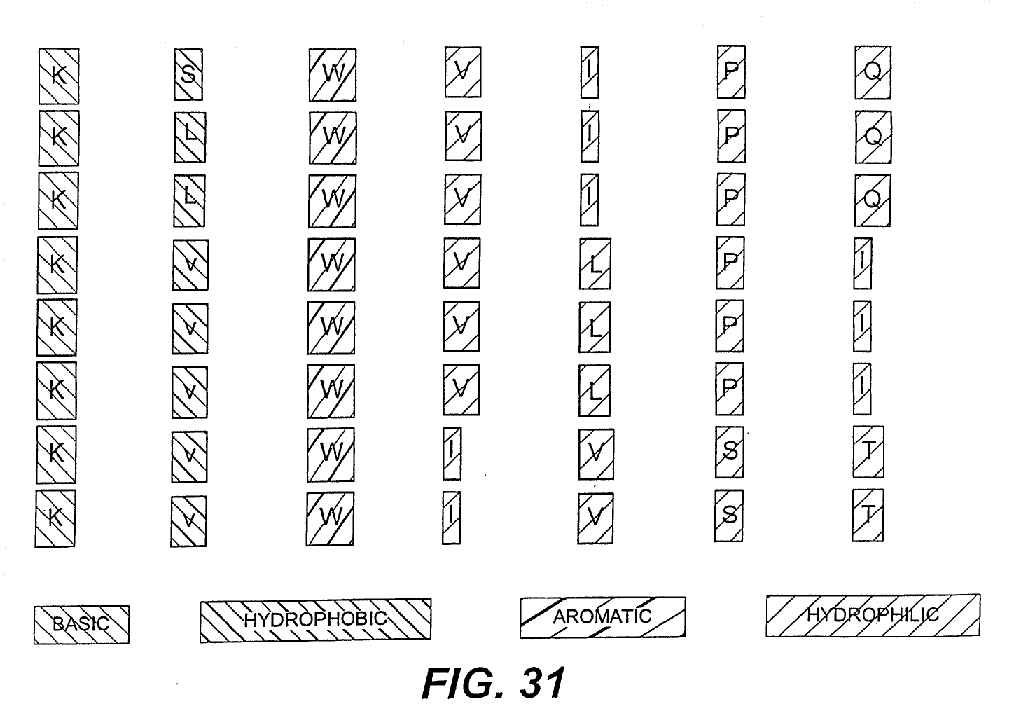

The phage display library identified a number of receptor binding peptides, as are shown in FIG. 31. These peptides are shown in FIG. 31, and as follows:

| | |
|---|---|
| Lys-Ser-Trp-Val-Ile-Pro-Gln; | (SEQ ID NO: 16) |
| Lys-Leu-Trp-Val-Ile-Pro-Gln; | (SEQ ID NO: 17) |
| Lys-Val-Trp-Val-Leu-Pro-Ile; | (SEQ ID NO: 18) |
| Lys-Val-Trp-Val-Leu-Ile-Pro; | (SEQ ID NO: 19) |
| Lys-Val-Trp-Val-Leu-Pro-Ile; | (SEQ ID NO: 18) |
| and | |
| Lys-Val-Trp-Ile-Val-Ser-Thr. | (SEQ ID NO: 20) |

Each line in FIG. 31 represents the one of the eight clones that were sequenced. The differences between the peptides are very small, with only conservative amino acid substitutions in terms of charge and class (for example, hydrophobic, aromatic, or hydrophilic).

Because these sequences are not linear sequences from TSP-1, it is believed they may represent an active site in the TSP-1 folded protein. Alternatively, they may represent a sequence from an additional protein that binds to angiocidin.

### Example 25

### Peptide Competition of TSP-1 and Angiocidin Binding

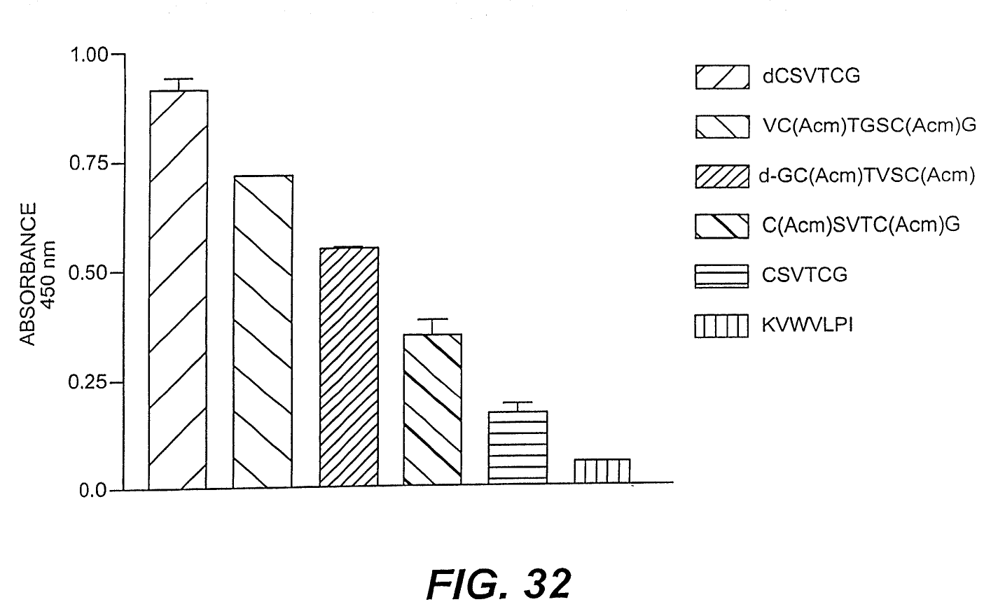

The avidin-biotin system discussed above was used to evaluate the competitive effect of various peptides on the binding of TSP-1 and angiocidin. The peptide Lys-Val-Trp-Val-Leu-Pro-IIe (SEQ ID NO: 18), identified by phage display as discussed in Example 24, inhibited the binding, as shown in FIG. 32. Additionally, the Cys-Ser-Val-Thr-Cys-Gly (SEQ ID NO: 1) peptide effectively inhibited binding. The more stable acetylated peptide Cys(Acm)-Ser-Val-Thr-Cys(Acm)-Gly (SEQ ID NO: 6) inhibited binding also. The mirror image acetylated peptide d-Gly-Cys(Acm)-Thr-Val-Ser-Cys(Acm) (SEQ ID NO: 23) inhibited binding most likely because it has the same stereoconfiguration. The scrambled peptide Val-Cys-Thr-Gly-Ser-Cys-Gly (SEQ ID NO: 21) and the d-orientation peptide d-Cys-Ser-Val-Thr-Cys-Gly (SEQ ID NO: 22) were used as negative controls.

### Example 26

### Effect of Angiocidin on the Viability of HAEC and HMVEC-L Cells

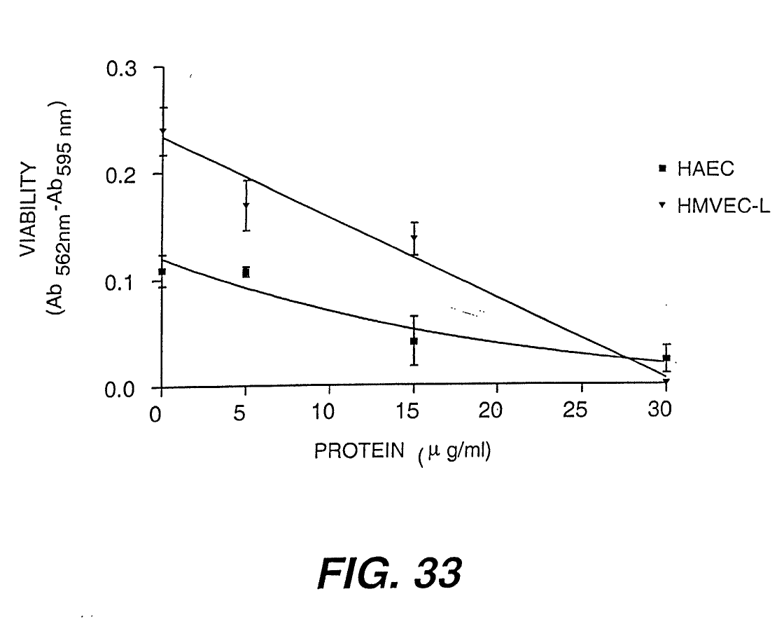

As discussed in Example 19 above, angiocidin was added to Human Aortic Endothelial Cells (HAEC) and Lung Human Microvascular Endothelial Cells (HMVEC-L). Angiocidin had a negative effect on the viability of both cell lines, as measured by the ALAMAR BLUE™ assay and shown in FIG. 33.

### Example 27

### Effect of Angiocidin and Fragments of Angiocidin on Viability of Bovine Aortic Endothelial Cells

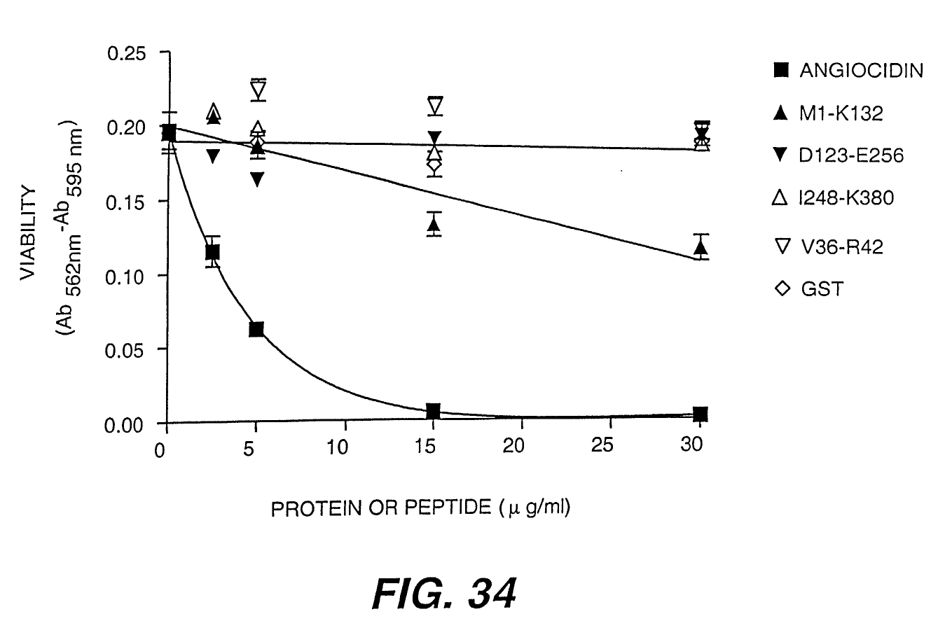

As discussed in Example 19 above, angiocidin was added to BAEC cells. Fragments of angiocidin were added as well. FIG. 34 shows that angiocidin and the amino terminal fragment Met1-Lys132 (expressed as a GST fusion protein, with GST coupled to the amino terminal side) inhibited cell viability. The middle domain of angiocidin and the carboxy terminus did not affect cell viability. GST was used as a negative control. V36-R42, the active site of the antisecretory factor, had no effect, illustrating that angiocidin plays a different role from antisecretory factor.

### Example 28

### Effect of Angiocidin on Growth of Lewis Lung Carcinoma Flank Tumors

Ten animals were subcutaneously injected in the flank with 106 Lewis lung carcinoma cells. Evaluation of flank tumors is a well recognized model for angiogenesis, because flank tumors are highly dependent on angiogenesis. O'Reilly, M. S., Angiostatin: A Novel Angiogenesis Inhibitor that Mediates the Suppression of Metastasis by a Lewis Lung Carcinoma, Cell 79: 315-28 (1994). After 9 days when a palpable tumor developed, mice were divided into two groups of 5 animals per group. One group of 5 mice were treated with an IV injection of 50 μg of angiocidin in Hepes buffered saline. The control group was treated with Hepes buffered saline. Mice were treated on days 1, 3, and 5 after the groups were divided, and sacrificed on day 7.

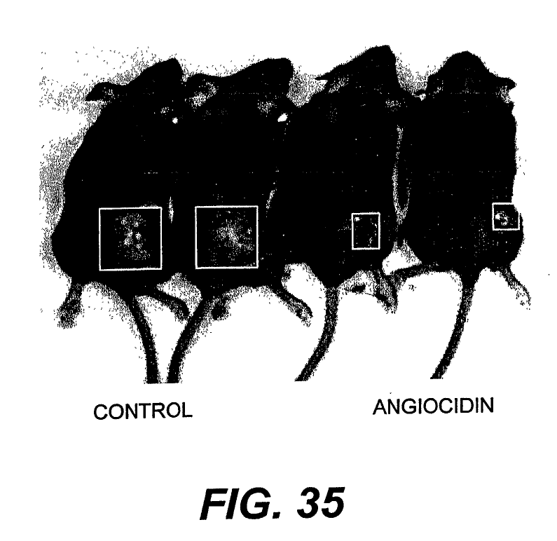

FIG. 35 shows the development of the flank tumors in the control and treatment group. The skin was removed to expose the tumor, which has been marked with a box. The tumors in the angiocidin mice were much smaller than the control mice. Additionally, the tumors in the angiocidin mice were soft, mushy, necrotic, and collapsed when pressure was applied. The tumors in the control mice were firm, fulminating, hard, heathy, and growing aggressively.

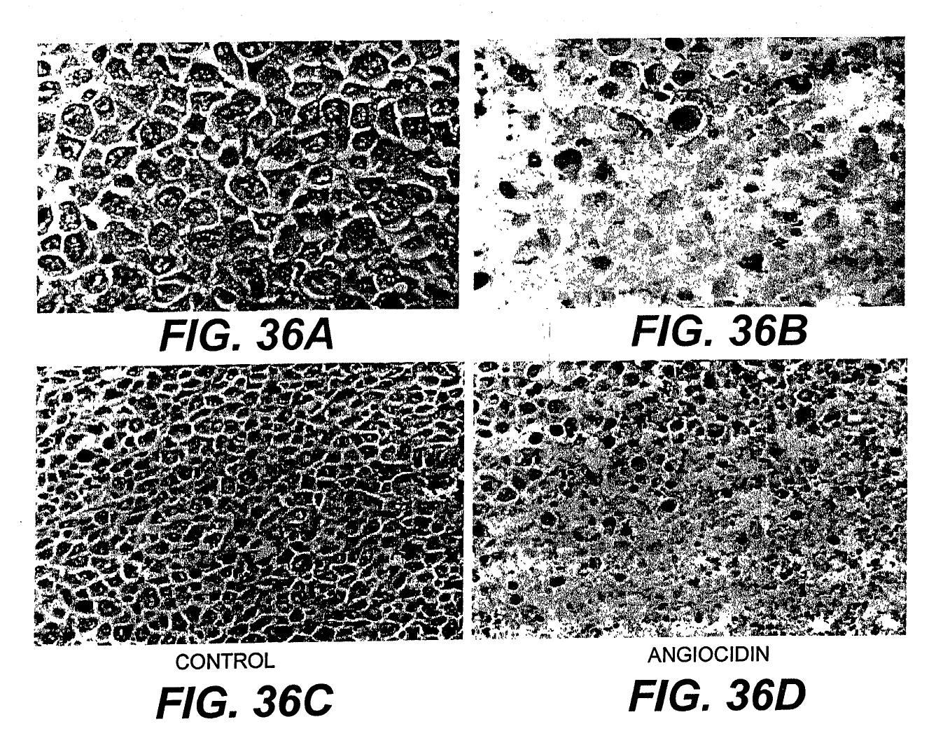

The tumors were embedded in paraffin and cut into 5 micron sections. The sections were stained with hemotoxylin and eosin. Hemotoxylin stains DNA blue, and eosin stains protein pink. FIG. 36 illustrates the difference between control (panels A and C) and angiocidin (panels B and D) treated cells. Panels A and B are at a magnification of 400×under a light microscope and panels C and D are at a magnification of 200×under a light microscope. The angiocidin-treated cells show significant necrosis and cell death.

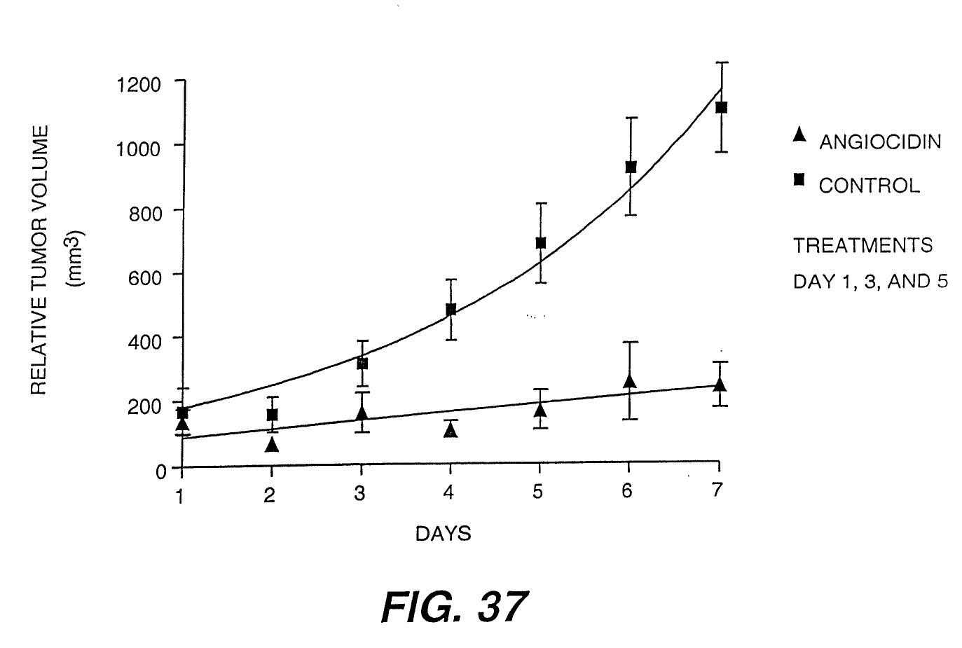

FIG. 37 shows the relative tumor volumes, measured as:

$$\frac{length \times {( width )}^{2}}{2} .$$

Measurements were taken for the entire 7 day treatment period. The control tumors grew exponentially, while the treatment tumors grew only slightly and at a linear rate. This shows that angiocidin had a significant effect on tumor growth and angiogenesis.

In combination with Example 20, this Example demonstrates that angiocidin directly affects angiogenesis, but does not affect the Lewis lung tumor cells themselves. Thus, the effect on tumor growth and tumor viability is a result of the effect on angiogenesis. Without proper blood supply, ensuring gas exchange and nutrients, a flank tumor greater than 2 mm³, which depends on vascularity, cannot survive.

### Example 29

### Survival Study of Mice Bearing Lewis Lung

Ten mice were injected with one million Lewis lung carcinoma tumor cells in an IV injection. After 3 days of incubation, the mice were divided into two groups. One group of five mice were treated with an IV injection of 50 μg of angiocidin in Hepes buffered saline. The control group of five mice was treated with Hepes buffered saline. Mice were treated on days 1, 3, 5, 7, and 9.

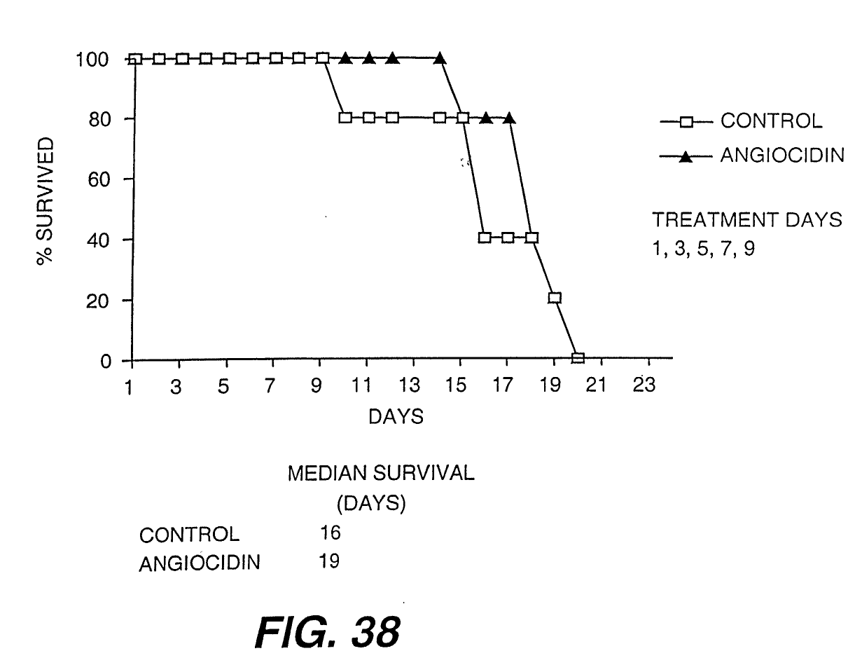

The survival of the two groups was evaluated. Even with only a moderate level of treatment (every other day and concluding on the 9^{th} day), the angiocidin group had a longer median survival period (19 days) than the control group (16 days), see FIG. 38.

The lung tumor is not a very good model for angiogenesis, because the lung is such a highly vascularized area and the tumor does not need to depend so significantly on additional vascularization. Nevertheless, this shows that angiocidin can effectively treat a cancerous lung tumor, extending lifespan in the process.

### Example 30

### Localization of Angiocidin in Human Breast Cancer Tissue

Human invasive breast carcinoma tumor samples, as well as benign and normal tissue samples as controls, were stained by immunoperoxidase staining. The samples were labeled with polyclonal antibodies against TSP-1 and angiocidin, then a secondary anybody against the first was added to the samples. The second antibody was conjugated to peroxidase, which when mixed with the substrate DAB, produces a brown color. All primary breast ductal carcinoma samples (n=11) stained positive for TSP-1 and angiocidin. In contrast, all benign lesions and normal breast tissue stained negative for TSP and angiocidin, with the exception of two fibrocystic breast samples with hyperplasia.

In the carcinoma samples, TSP-1 stained in the dense stromal collagen adjacent to the tumor, whereas angiocidin stained in the tumor cells. These results show increasing expression of TSP-1 and angiocidin in ductal epithelium correlates with neoplastic transformation.

### Example 31

### Localization of Angiocidin in Human Head & Neck Tumor Tissue

Human head and neck tumor samples were stained with hematoxylin, eosin, and angiocidin antibody. The stained tumors were analyzed by a computer video microscope that emits light at a single wavelength (620 nm) and measures the optical density of the stained tumor fields. Adjacent normal mucosa were also analyzed for every specimen. The objective antibody threshold for specific staining was defined for each specimen by analyzing the negative control section (control IgG) and subtracting this value from the angiocidin stained fields. In this way, an accurate quantitation of the percent positive receptor-staining cells was obtained. Using the image analysis technique, we found that those patients with a high positive stain score had a high microvessel density and died from metastatic disease within 12 months of initial treatment. Patients with a low positive stain score had low microvessel counts and remained disease-free for at least 2 years. Data are presented in Table 3, below.

TABLE 3
Head and Neck Tumors

| | Histologic | | | |
|---|---|---|---|---|
| | Differ- | Angiocidin | Angiogenesis | 2 year |
| Site | entiation | Density | (vessels/mm²) | Survival |
| Tonsil | Moderate | 5 | 52 | Alive |
| Floor of | Poor | 5 | 24 | Alive |
| Mouth | | | | |
| Pharynx | Poor | 9 | 15 | Alive |
| Tongue | Moderate | 14 | 10 | Alive |
| Buccal | Well | 73 | 140 | Dead |
| Tongue | Poor | 82 | 213 | Dead |

### Example 32

### Endotoxin Study

Angiocidin samples were evaluated for the presence of endotoxin to ensure that there was no contaminating endotoxin affecting the cell culture using a timed gel formation endotoxin kit available from Sigma (St. Louis, Mo.). The angiocidin sample gave a measurement of 0.0076 picogram endotoxin/microgram of protein. Levels below 1 nanogram are considered safe for tissue culture. Therefore, it is evident that the angiocidin itself is having the inhibitory effect on cell viability.

### Example 33

### Viability Study

His tagged angiocidin was compared to his tagged control GST protein to show that the his tag does not have any effect on cell viability. Bovine aortic endothelial cells (BAEC) were cultured overnight in serum-free media containing either 37 μg/ml his-tagged angiocidin or his-tagged GST. Both angiocidin and GST were expressed in bacteria transformed with the pTrcHisA expression vector and purified on nickel affinity chromatography under non-denaturing conditions. Viability was measured by the ALAMAR BLUE™ assay.

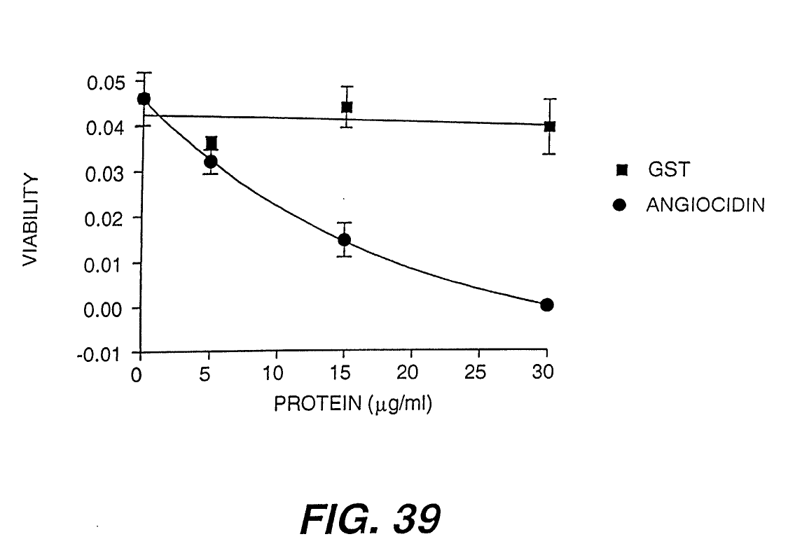

FIG. 39 shows that the angiocidin had a dose-dependent effect on cell viability, with viability decreasing with increasing concentrations of angiocidin. GST did not have any effect on cell viability. This study shows that under non-denaturing conditions, i.e., closer to physiological conditions than denaturing conditions, the his tag does not have any effect on cell viability.

### Example 34

### Effect of Anti-Angiocidin Antibody on Angiocidin-Mediated Inhibition of BAEC Viability

This study examined the effect of anti-angiocidin antibody on angiocidin-mediated inhibition of BAEC viability. BAEC were cultured overnight in serum-free media containing either 5 μg/ml angiocidin, 5 μg/ml angiocidin plus 100 μg/ml control IgG, or 5 μg/ml angiocidin plus 100 μg/ml anti-angiocidin IgG. Viability was measured using the ALAMAR BLUE™ assay, described above.

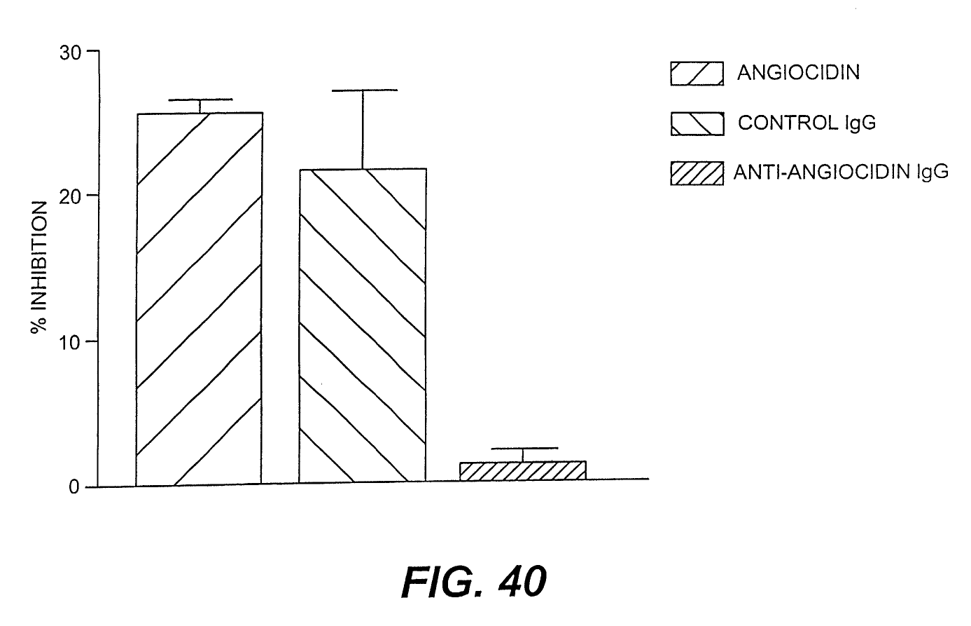

FIG. 40 demonstrates that the anti-angiocidin IgG virtually eliminated all of the angiocidin inhibition of BAEC viability. Control IgG did not have any notable effect. This example shows that the effect of angiocidin is specific and not due to any contamination in the preparations.

### Example 35

### Effect of Angiocidin on Adhesion of BAEC to a Substrate

This example evaluates the effect of angiocidin on adhesion of BAEC to a substrate. Cells in the treatment group were pretreated with angiocidin (37 μg/ml). Cells in the control group were not pretreated. Cells (50,000) were immediately plated on microtiter wells coated with 2 μg of either fibronectin, TSP-1, or BSA. Fibronectin is a strong extracellular matrix protein that attracts BAEC and serves as a positive control, whereas BSA is not an adhesion protein and serves as a negative control. After 30 minutes non-adherent cells were aspirated, wells washed with PBS, fixed with 2.5% glutaraldehyde, stained with 2% Giemsa, and the number of adherent cells per 1 mm² counted.

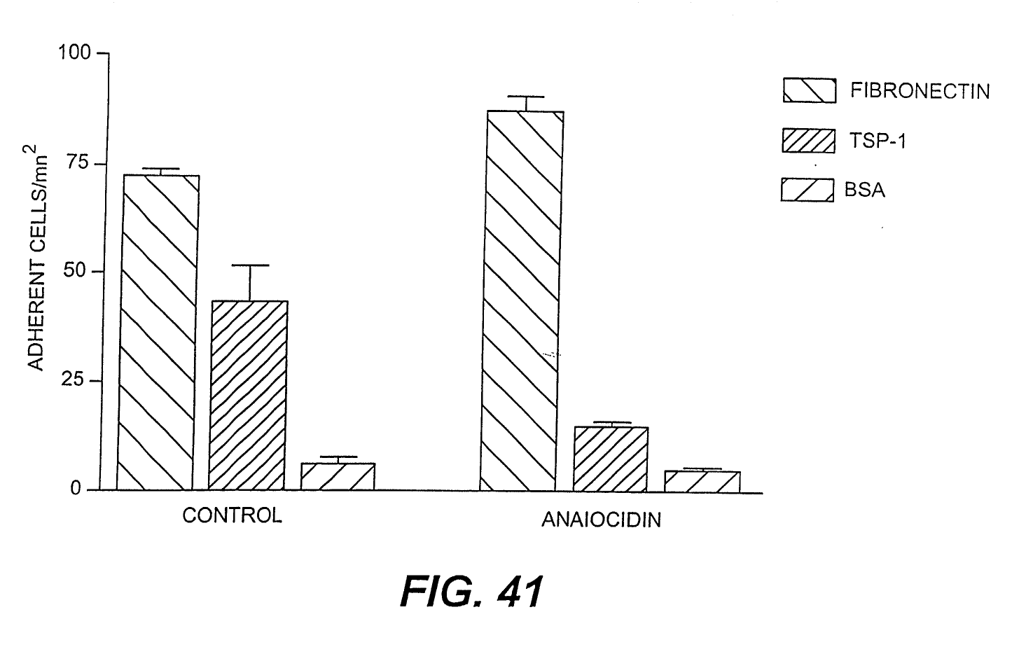

FIG. 41 illustrates the results of this study. In the cells that were not treated with angiocidin, the fibronectin group showed very strong adhesion and the TSP-1 group showed strong adhesion. When the cells were treated with angiocidin, the adherence of the cells in the fibronectin group remained the same (very strongly adherent), but the cells in the TSP-1 group had a sharp drop off in adherence.

This shows that addition of angiocidin significantly reduced the adhesion of BAEC to the TSP-1 coated plates, but not to the positive control fibronectin plates. Angiocidin has a specific interaction with TSP-1, disrupting its adhesive mechanism.

### Example 36

### Functionality of the Amino Terminal and Carboxy Terminal Portions of Angiocidin

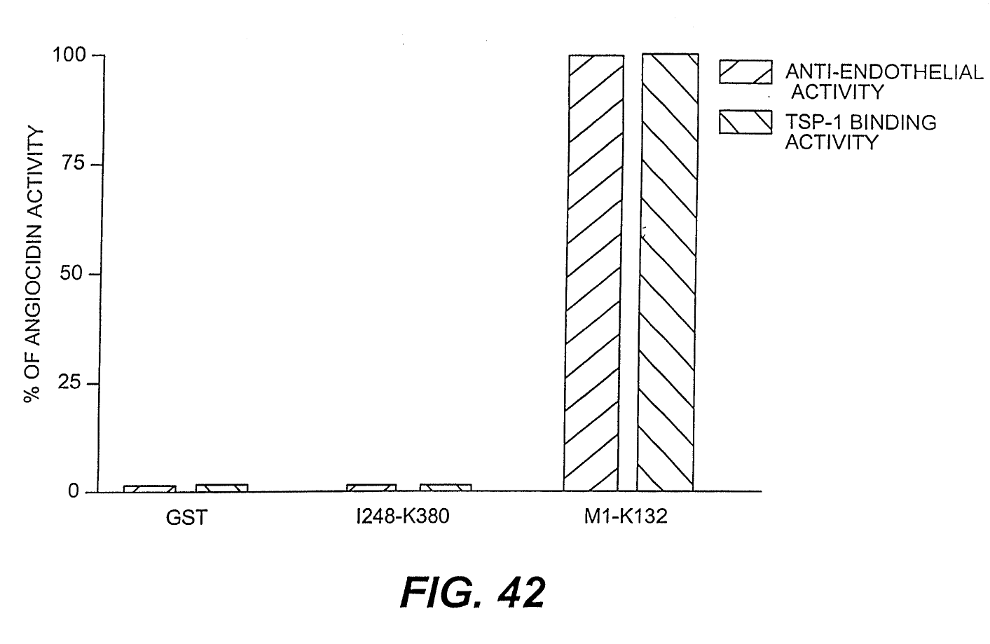

This study examines the amino terminal (Met1-Lys132) and carboxy terminal (IIe248-Lys380) portions of angiocidin (SEQ ID NOS: 24 and 25, respectively). The binding of undenatured recombinant angiocidin fragments was compared to full length angiocidin. GST was used as a negative control. Binding was evaluated using an optical binding method that uses a cuvette to which TSP-1 is covalently coupled. A laser beam was used to detect whether the test protein (fragments, angiocidin, or GST) is bound to the TSP-1 derivatized cuvette surface. The cuvette was derivatized with 1 μg of TSP-1. The cuvette surfaces were blocked with a 1% BSA solution to prevent nonspecific binding. The test proteins were added at a concentration of 10 nm in a PBS buffer. Results, shown in FIG. 42, demonstrate that both angiocidin and its amino terminal fragment (Met1-Lys132) show very similar binding at the nano molar range. FIG. 42 shows the percent activity compared to angiocidin. Both GST and the carboxy terminal fragment show no binding activities.

### Example 37

### Functionality of the Amino Terminal and Carboxy Terminal Portions of Angiocidin

This study examines the amino terminal (Met1-Lys132) and carboxy terminal (IIe248-Lys380) portions of angiocidin (SEQ ID NOS: 24 and 25, respectively). The anti-endothelial activity of the fragments was compared to that of the full length angiocidin protein.

The endothelial cells (BAEC) were incubated overnight 37 μg/ml of the angiocidin, fragments, and GST. Viability was measured using the ALAMAR BLUE™ assay.

These results are also shown in FIG. 42, as a percentage of anti-endothelial activity of the fragments compared to angiocidin. This shows that the amino terminal end has the same anti-endothelial activity as the full length angiocidin. Furthermore, the binding and anti-endothelial activity of the amino terminal region correlate very well.

We claim:

1. A purified receptor protein having specific binding affinity for the Cys-Ser-Val-Thr-Cys-Gly (SEQ ID NO: 1)-specific region of thrombospondin (TSP-1).

2. The receptor of claim 1, comprising a sequence selected from the group consisting of SEQ ID NO: 2 and SEQ ID NO: 3, and fragments and mutations of SEQ ID NO. 2 and SEQ ID NO. 3.

3. The receptor of claim 2, wherein the fragment comprises SEQ ID NO. 24, and fragments and mutations of SEQ ID NO. 24.

4. A method of treating a patient with an antibody that inhibits thrombospondin activity comprising the steps of isolating the receptor of claim 1, generating antibodies to the receptor, and using the antibodies to treat the patient.

5. A method of treating a patient with an antibody that mimics thrombospondin activity comprising the steps of isolating the receptor of claim 1, generating antibodies to the receptor, and using the antibodies to treat the patient.

6. A method of treating a patient with a ligand that inhibits thrombospondin activity comprising the steps of isolating the receptor of claim 1, generating a ligand to the receptor, and using the ligand to treat the patient.

7. A method of detecting malignant cancer comprising the steps of measuring the presence of the receptor of claim 1, and determining whether malignant cancer is present.

8. A method of treating a patient with a ligand that mimics thrombospondin activity comprising isolating the receptor of claim 1, generating a ligand to the receptor, and using the ligand to treat the patient.

9. A method of treating a patient with the receptor of claim 1 comprising administering the receptor to the patient and allowing the receptor to competitively inhibit thrombospondin activity.

10. The method of claim 8, wherein the method of treatment inhibits or reverses angiogenesis.

11. The method of claim 8, wherein the method of treatment inhibits, prevents, or reverses tumor growth.

12. The method of claim 8, wherein the method extends the life of the patient.

13. A method of treating a patient with a fragment of the receptor of claim 1 comprising the steps of administering a fragment of the receptor is administered to the patient and allowing the fragment to competitively inhibit thrombospondin activity.

14. A method of diagnosing or determining the prognosis of a patient with cancer comprising the steps of determining the level of receptor of claim 1 and evaluating the level against known values for metastatic and nonmetastatic tumors.

15. A composition for treating cancer comprising a chemotherapy drug linked to a targeting moiety, wherein the targeting moiety is selected from the group consisting of an antibody directed to the receptor of claim 1 or a ligand directed to the receptor of claim 1.

16. A composition for treating cancer comprising a radioactive moiety linked to a targeting moiety, wherein the targeting moiety is selected from the group consisting of an antibody directed to the receptor of claim 1 or a ligand directed to the receptor of claim 1.

17. A method for treating cancer comprising administering a therapeutically effective amount of the composition of claim 16, optionally in a pharmaceutically acceptable carrier, allowing the targeting moiety to target the cancer, and allowing the radioactive moiety to treat the cancer.

18. A composition for radiological detection of cancer, diagnosis of cancer, and quantification of therapeutic response to treatment of cancer comprising a radioactive moiety linked to a targeting moiety, wherein the targeting moiety is selected from the group consisting of an antibody directed to the receptor of claim 1 or a ligand directed to the receptor of claim 1.

19. A method for radiological detection of cancer, diagnosis of cancer, and quantification of therapeutic response to treatment of cancer comprising administering a effective amount of the composition of claim 18, optionally in a pharmaceutically acceptable carrier, allowing the targeting moiety to target the cancer, allowing the radioactive moiety to label the cancer, and detecting the cancer, diagnosing the cancer, or quantifying the therapeutic response to treatment of cancer.

20. A composition for MRI detection of cancer, diagnosis of cancer, and quantification of therapeutic response to treatment of cancer comprising an MRI enhancing agent linked to a targeting moiety, wherein the targeting moiety is selected from the group consisting of an antibody directed to the receptor of claim 1 or a ligand directed to the receptor of claim 1.

21. The composition of claim 20, wherein the MRI enhancing agent is selected from the group consisting of gadolinium, manganese, and iron.

22. A method of MRI detection of cancer, diagnosis of cancer, and quantification of therapeutic response to treatment of cancer comprising administering an effective amount of the composition of claim 20, optionally in a pharmaceutically acceptable carrier, allowing the targeting moiety to target the cancer, using MRI to detect the cancer, diagnose the cancer, or quantify the therapeutic response of the cancer, and allowing the MRI enhancing agent to enhance the MRI.

23. A biomedical device comprising a means to remove cells, wherein the cell removing means is linked to a targeting moiety and the targeting moiety is selected from the group consisting of an antibody directed to the receptor of claim 1 or a ligand directed to the receptor of claim 1.

24. A method of designing a drug to mimic or inhibit thrombospondin activity comprising the steps of developing a candidate drug and evaluating its binding to the receptor of claim 1.

25. A method of decreasing endothelial cell viability comprising administering a pharmaceutically acceptable amount of the purified receptor protein of claim 1 and allowing it to interact with the endothelial cell to decrease endothelial cell viability.

26. A method of decreasing cell adhesion activity comprising administering a pharmaceutically acceptable amount of the purified receptor protein of claim 1 and allowing it to interact with the cell to decrease cell adhesion activity.

27. A method of treating a patient comprising administering to the patient a purified receptor protein having specific binding affinity for the Cys-Ser-Val-Thr-Cys-Gly (SEQ ID NO: 1)-specific region of thrombospondin (TSP-1), wherein the receptor protein is reactive with antibodies raised against SEQ ID NO:24, and allowing the receptor protein to competitively inhibit thrombospondin activity.

28. The method of claim 27, wherein the method of treatment inhibits or reverses angiogenesis.

29. The method of claim 27, wherein the method of treatment inhibits, prevents, or reverses tumor growth.

30. The method of claim 27, wherein the method extends the life of the patient.

31. The method of claim 27, wherein the method decreases endothelial cell viability.

32. The method of claim 27, wherein the method decreases cell adhesion activity.

33. The method of claim 27, wherein the receptor protein comprises a sequence chosen from SEQ ID NO: 2, SEQ ID NO: 3, fragments of SEQ ID NO:2 and SEQ ID NO:3, and mutations of SEQ ID NO:2 and SEQ ID NO:3.

34. The method of claim 27, wherein the receptor protein comprises a sequence chosen from SEQ ID NO: 24, fragments of SEQ ID NO:24, and mutations of SEQ ID NO:24.
